(19) United States
(12) Patent Application Publication
(10) Pub. No.: US 2026/0192176 A1
(43) Pub. Date: Jul. 9, 2026

(54) SYSTEMS AND METHODS FOR FACILITATING SITUATION AWARENESS FOR A PHYSICAL ACTIVITY

(71) Applicant: Neotericc LLC, LONE TREE, CO (US)

(72) Inventor: Patrick L. Carter, Lone Tree, CO (US)

(73) Assignee: Neotericc LLC, LONE TREE, CO (US)

(21) Appl. No.: 19/435,932

(22) Filed: Dec. 30, 2025

Related U.S. Application Data
(60) Provisional application No. 63/739,818, filed on Dec. 30, 2024.

(51) Int. Cl. A63B 71/06; A63B 24/00

(52) U.S. Cl. CPC A63B 71/0622; A63B 24/0006; A63B 2024/0015; A63B 2024/0025; A63B 2024/0068; A63B 2220/05; A63B 2220/62; A63B 2220/806

(57) ABSTRACT

The present disclosure provides a method for facilitating situation awareness for a physical activity. Further, the method may include receiving one or more content data representing one or more contents associated with one or more physical activities. Further, the method may include identifying a situation data using one or more temporal artificial intelligence (AI) models. Further, the situation data may represent one or more situations associated with the one or more physical activities. Further, the method may include generating an identifier data based on the situation data. Further, the identifier data represents one or more identifiers for the one or more situations. Further, the one or more identifiers represent a position of the one or more situations in a sequence corresponding to the one or more physical activities. Further, the method may include storing each of the one or more content data and the identifier data.

### REFERENCE TO RELATED APPLICATIONS

This application claims the benefit of U.S. Provisional Patent Application No. 63/739,818, titled “METHODS AND SYSTEMS OF PROVISIONING A CONTEXTUAL FEEDBACK OF A PHYSICAL ACTIVITY”, filed on Dec. 30, 2024, which is incorporated by reference herein in its entirety.

### FIELD OF DISCLOSURE

The present disclosure relates to the field of data processing. More specifically, the present disclosure relates to methods and systems for facilitating situation awareness for a physical activity.

### BACKGROUND

The field of computerized analysis of physical activities, including techniques for interpreting human motion, evaluating athletic performance, and generating actionable feedback using computational systems, is of increasing importance as modern training environments, competitive sports programs, and remote coaching platforms rely heavily on objective, data-driven insights into biomechanical movements and physical performance. Accurate interpretation of motion sequences and timely delivery of feedback may significantly influence athletic development, injury prevention, rehabilitation efficiency, and overall performance optimization. As sports, fitness, and movement analytics continue to integrate advanced computational capabilities, there is a growing demand for systems that may analyze complex physical activities in real time and provide meaningful, contextually aware insights.

In many applications, a desirable aspect is to achieve automated, reliable, and comprehensive evaluation of physical activity in a manner that accounts for the temporal evolution of movement, contextual factors surrounding the activity, and the dynamic characteristics of both athlete biomechanics and associated sport objects. Achieving the said objective requires the ability to process high-volume sequential data, interpret transitions within movement sequences, identify activity patterns, and produce feedback that reflects real-world conditions under which the activity is performed.

However, existing or known systems and methods face several challenges in achieving such an objective. Many conventional approaches are limited in their ability to accurately interpret physical activities that unfold over time and may struggle to detect subtle transitions between different phases of a movement. Some existing systems may be confined to static or frame-based analysis, which may not capture dependencies across sequences or may fail to represent the nuances of biomechanical behavior. Other approaches may rely on separate mechanisms or isolated sensor systems to track athlete movement and sport-object behavior, resulting in fragmented or incomplete performance evaluations. Systems that attempt to process continuous data streams may experience latency, reduced throughput, frame loss, or inconsistencies in analysis due to computational bottlenecks. Additional difficulties may arise when integrating multiple types of data, such as spatial, temporal, or contextual information, which may lead to misalignment or incomplete interpretation of the activity.

Furthermore, existing techniques may not provide meaningful customization or adaptability in generating feedback, particularly in scenarios involving different environmental conditions, athlete fatigue levels, or variations in sport-specific contexts. Some systems may lack the ability to deliver partial or preliminary analysis in real time, limiting opportunities for immediate correction or intervention by coaches or users. Many approaches may also struggle to maintain efficiency at scale, handle data variability, or adapt to differences in athletic style, experience level, or situational complexity.

Therefore, there is a need for improved methods and systems for facilitating situation awareness for a physical activity that may overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method for facilitating situation awareness for a physical activity. Further, the method may include receiving, using a communication device, one or more content data from one or more content source devices. Further, the one or more content data represent one or more contents associated with one or more physical activities. Further, the method may include identifying, using a processing device, a situation data using one or more temporal artificial intelligence (AI) models based on the one or more content data. Further, the situation may data represent one or more situations associated with the one or more physical activities. Further, the method may include generating, using the processing device, an identifier data based on the situation data. Further, the identifier data represents one or more identifiers for the one or more situations. Further, the one or more identifiers represent a position of the one or more situations in a sequence corresponding to the one or more physical activities. Further, the method may include storing, using a storage device, each of the one or more content data and the identifier data.

The present disclosure provides a system for facilitating situation awareness for a physical activity. Further, the system may include a communication device which may be configured for receiving one or more content data from one or more content source devices. Further, the one or more content data represent one or more contents associated with one or more physical activities. Further, the system may include a processing device communicatively coupled with the communication device. Further, the processing device may be configured for identifying a situation data using one or more temporal artificial intelligence (AI) models based on the one or more content data. Further, the situation may data represent one or more situations associated with the one or more physical activities. Further, the processing device may be configured for generating an identifier data based on the situation data. Further, the identifier data represents one or more identifiers for the one or more situations. Further, the one or more identifiers represent a position of the one or more situations in a sequence corresponding to the one or more physical activities. Further, the system may include a storage device communicatively coupled with the processing device. Further, the storage device may be configured for storing each of the one or more content data and the identifier data.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

The drawings presented with this disclosure may illustrate representative and non-limiting arrangements of hardware components, software modules, artificial intelligence subsystems, machine learning architectures, data processing pipelines, user interfaces, network topologies, and memory arrangements that may be used to understand embodiments of the present subject matter. They may depict functional or conceptual layouts intended to facilitate explanation of the disclosed principles. The geometric appearance, dimensional proportions, ordering, grouping, and naming of elements within the drawings are not intended to imply any restriction on implementation. The drawings may schematically portray computing environments containing client devices, servers, distributed computing clusters, communication networks, storage systems, or artificial intelligence models arranged for training, inference, or combined operations. The drawings may include simplified symbolic representations of algorithmic processes, workflows, blocks, or modules; such symbolic representations are treated as abstractions of underlying hardware and software operations rather than literal structural requirements. Similarly, lines connecting components may represent logical associations, communication pathways, or data relationships rather than any specific physical wiring or layout. These figures may also illustrate non-exhaustive examples of operational stages, sequencing, or interactions among artificial intelligence components such as encoders, decoders, generators, discriminators, featurizers, transformers, or safety-validation modules. Any specific combination or configuration shown is presented for explanatory clarity only. Additional drawings, alternative views, or more granular depictions may be used without affecting the scope of the claims.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 is a block diagram illustrating a machine-learning system 300 for implementing various embodiments of this disclosure, in accordance with some embodiments.

FIG. 4 illustrates a flowchart of a method 400 for facilitating situation awareness for a physical activity, in accordance with some embodiments.

FIG. 5 illustrates a flowchart of a method 500 for facilitating situation awareness for a physical activity including generating, using the processing device 1004, at least one feedback data, in accordance with some embodiments.

FIG. 6 illustrates a flowchart of a method 600 for facilitating situation awareness for a physical activity including generating, using the processing device 1004, an overlaid video stream data, in accordance with some embodiments.

FIG. 7 illustrates a flowchart of a method 700 for facilitating situation awareness for a physical activity including identifying, using the processing device 1004, an activity data, in accordance with some embodiments.

FIG. 8 illustrates a flowchart of a method 800 for facilitating situation awareness for a physical activity including determining, using the processing device 1004, at least one information of the at least one equipment, in accordance with some embodiments.

FIG. 9 illustrates a flowchart of a method 900 for facilitating situation awareness for a physical activity including analyzing, using the processing device 1004, the temporal order, in accordance with some embodiments.

FIG. 10 illustrates a block diagram of a system 1000 for facilitating situation awareness for a physical activity, in accordance with some embodiments.

FIG. 11 illustrates a block diagram of the system 1000 for facilitating situation awareness for a physical activity including at least one coach device 1102, in accordance with some embodiments.

FIG. 12 illustrates a flowchart of a method 1200 for facilitating situation awareness for a physical activity including obtaining, using the processing device 1004, at least one trained temporal artificial intelligence (AI) model, in accordance with some embodiments.

FIG. 13 illustrates a flowchart of a method 1300 for facilitating situation awareness for a physical activity including generating, using the processing device 1004, at least one anonymized feedback data, in accordance with some embodiments.

FIG. 14 illustrates a flowchart of a method 1400 for facilitating situation awareness for a physical activity including analyzing, using the processing device 1004, the input data, in accordance with some embodiments.

FIG. 15 illustrates a flowchart of a method 1500 for facilitating situation awareness for a physical activity including identifying, using the processing device 1004, a transition data representing a phase transition within the temporal pattern data, in accordance with some embodiments.

FIG. 16 illustrates a flowchart of a method 1600 for facilitating situation awareness for a physical activity including computing, using the processing device 1004, at least one performance metric for the at least one entity, in accordance with some embodiments.

FIG. 17 illustrates a flowchart of a method 1700 for facilitating situation awareness for a physical activity including determining, using the processing device 1004, a situation condition data, in accordance with some embodiments.

FIG. 18 illustrates a flowchart of a method 1800 for facilitating situation awareness for a physical activity including generating, using the processing device 1004, at least one enhanced content data, in accordance with some embodiments.

FIG. 19 illustrates a user device 1902 configured for presenting the one or more phases 1904 associated with the one or more physical activities, in accordance with some embodiments.

FIG. 20 illustrates a spatial-temporal mechanics of a player playing hockey, in accordance with some embodiments.

FIG. 21 illustrates the embedding of the one or more identifiers within the visual sequence of the one or more physical activities, in accordance with some embodiments.

FIG. 22 illustrates a flowchart of a method 2200 of provisioning a contextual feedback of a physical activity, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

The detailed description that follows may provide a framework for describing computer-implemented systems, artificial intelligence systems, distributed learning infrastructures, data processing pipelines, and hardware and software arrangements suitable for implementing embodiments shown in the drawings. Terms such as processing, computing, determining, or generating refer to actions performed by computing systems or electronic devices that manipulate data represented as physical signals, stored values, or encoded information within registers, memory structures, and storage devices.

The present disclosure contemplates implementations involving artificial intelligence, machine learning, distributed computation, and computer-implemented systems operating upon data represented as physical electronic or optical signals. Descriptions of processing, analyzing, determining, transforming, encoding, decoding, generating, inferring, synthesizing, modifying, storing, retrieving, ranking, filtering, validating, classifying, or otherwise manipulating information are to be understood as referring to the actions of computing systems, electronic devices, or computational circuits that manipulate such signals in memory elements, registers, buffers, or storage media. These operations may be performed by general-purpose processors, specialized processors, machine learning accelerators, or combinations thereof.

The disclosure contemplates implementations in which artificial intelligence systems perform perception, synthesis, inference, prediction, or generation of information using models whose configurations may evolve based on training, feedback, or adaptive learning processes. A model may initially be configured with a set of parameters and architectural structures that define its behavior, and this configuration may change automatically as the model encounters training inputs, validation data, reference data, or instructor-provided feedback. A machine learning model may modify its internal state through optimization techniques, gradient updates, reinforcement signals, vector transformations, attention mechanisms, latent variable adjustments, embedding refinements, or other learning operations executed electronically. Such modifications may occur over extended cycles, partial cycles, or continual learning sequences without explicit intervention by a human.

The disclosure contemplates systems involving data ingestion pipelines that gather input from sources including but not limited to sensor signals, event streams, text data, image data, audio data, video data, structured and unstructured repositories, application logs, telemetric feeds, network services, or human-generated content. Ingestion functions may include filtering, normalization, augmentation, segmentation, batching, tokenization, windowing, compression, encryption, decryption, hashing, deduplication, contextualization, and mapping to internal formats. Intermediate components may transform this data into derived representations, including embeddings, latent encodings, feature tensors, multi-modal joint representations, or contextual vectors suitable for use by downstream modeling engines. These transformations may be performed using neural networks, statistical encoders, dimensionality-reduction algorithms, or hybrid computational modules.

The disclosure contemplates machine learning systems that may employ advanced architectures such as transformer networks, encoder-decoder stacks, mixture-of-experts structures, diffusion models, recurrent networks, convolutional hierarchies, attention-based models, retrieval-augmented architectures, cross-modal alignment engines, graph neural networks, probabilistic models, auto-encoding frameworks, or hybrid symbolic-neural systems. Such models may implement deep layers configured to perform operations including attention calculations, feed-forward projections, gating operations, positional encoding, normalization steps, multi-head routing, sequential decoding, or latent pathway selection. Multi-modal systems may combine textual, visual, auditory, sensory, or structured inputs within joint representational spaces. Embeddings may be learned from large corpora or multi-modal datasets and may encode semantic, syntactic, structural, temporal, spatial, or contextual relationships across modalities. These embeddings may be dynamically updated as the system encounters new information, thereby improving consistency, expressiveness, or alignment with real-world contexts.

The disclosure contemplates training processes that may involve supervised learning, unsupervised learning, semi-supervised learning, self-supervised learning, reinforcement learning, preference optimization, curriculum-based learning, active learning, or continual learning. Training operations may include forward passes through the model, backward propagation of gradients, update steps using optimization algorithms, adaptive learning-rate scheduling, regularization steps, loss-function evaluation, and check pointing of intermediate states. Training datasets may include real-world data, synthetic data, simulated data, augmented data, or mixtures thereof. Validation procedures may evaluate performance metrics, generalization behavior, safety constraints, or compliance with domain-specific criteria. In some implementations, refinement cycles may incorporate human-in-the-loop interventions, reward model shaping, safety evaluator feedback, or guided corrections.

The disclosure contemplates distributed or federated execution in which computation is partitioned across multiple hardware devices, regions, or clusters. Certain operations may occur at edge devices for low latency, while others may be delegated to remote servers, cloud clusters, datacenters, or specialized compute fabrics. Components may communicate over wired or wireless networks supporting data exchange, synchronization, replication, or model-state updates. Distributed learning processes may synchronize gradients, coordinate model versions, merge updates across shards, or exchange activation values within parallel training regimes. Distributed inference may involve routing requests across replicas, balancing load through orchestration layers, or selecting model pathways dynamically. Network connections may include encryption, authentication, secure session management, or routing protocols appropriate for maintaining privacy, integrity, or availability.

The disclosure contemplates orchestration layers capable of managing complex workflows involving model invocation, tool invocation, external data retrieval, decision routing, fallback selection, multi-model aggregation, post-processing evaluation, or safety governance. Orchestration environments may evaluate contextual signals, metadata, user characteristics, or policy constraints to determine which models, subsystems, or computational branches should be executed. Such environments may dynamically alter execution pathways based on estimated performance, resource availability, model confidence, safety risk, or real-time system health. Post-processing components may evaluate generated outputs for compliance with content policies, statutory requirements, operational constraints, or domain-specific decision rules.

The disclosure contemplates safety-oriented components that evaluate model outputs or intermediate representations for consistency with safety criteria, quality thresholds, regulatory considerations, factual accuracy constraints, domain restrictions, or alignment requirements. Safety modules may employ auxiliary models, discriminators, rule sets, statistical detectors, confidence estimators, or hybrid evaluators to identify undesirable outputs. These modules may trigger remediation actions including output modification, output rejection, re-routing through alternate inference pathways, invocation of corrective models, or escalation for human review. Safety processes may incorporate real-time validation, contextual scoring, adversarial robustness analysis, anomaly detection, or controlled generation constraints.

The disclosure contemplates governance structures including policy managers, audit loggers, compliance trackers, version controllers, and provenance systems that associate model outputs with contextual metadata, historical signals, update events, training sources, or safety evaluations. Systems may maintain lineage records documenting which model version, configuration state, or training dataset contributed to an outcome. Governance modules may ensure that system behavior aligns with formal requirements such as fairness principles, legal obligations, industry standards, or institutional guidelines.

The disclosure contemplates storage and memory systems capable of storing model parameters, datasets, embeddings, logs, metrics, checkpoints, execution traces, and auxiliary information used to configure or interpret model behavior. These storage systems may include magnetic media, semiconductor memory, optical media, solid-state arrays, distributed storage fabrics, or hybrid memory hierarchies. Storage media may contain instructions, configurations, or data structures that, when accessed by a computing device, configure that device to carry out the operations described herein. Such media may include executables, bytecode, machine code, firmware, microcode, program modules, configuration files, architectural descriptors, or schema definitions.

The disclosure contemplates user interfaces that permit human operators to view model outputs, initiate tasks, modify configurations, inspect metrics, interact with logs, evaluate safety signals, or guide system adaptation. Interfaces may be multimodal and may support textual input, speech commands, visual interaction, gesture control, or programmatic invocation through APIs. Administrative interfaces may allow for reviewing system performance, tuning operational thresholds, enabling or disabling features, monitoring resource use, examining generated content, or initiating refinement workflows.

The disclosure contemplates systems in which instructions are executed entirely on a single device, partially on multiple devices, or cooperatively across remote and local environments. Code may execute directly on hardware, within firmware, inside virtual machines, inside containers, or through any combination of software and hardware interactions. Computational instructions may be stored locally, transferred via communication networks, or streamed from remote systems. Implementations may involve software executing on general-purpose processors, specialized logic circuits performing equivalent functions, or hybrid mechanisms that combine hardware acceleration with software guidance.

Interpretation of terms in this disclosure is governed by principles commonly applied by persons of ordinary skill in the relevant field. Technical and scientific terms used herein should be understood in a manner consistent with their usage in the field of artificial intelligence, machine learning, computing, networking, data storage, or any related discipline. Terms describing functionality should not be interpreted as strictly structural unless explicitly stated. Phrases such as configured to, adapted to, operable to, or capable of indicate permissible functionality rather than structural limitations. Terms such as a or an encompass one or more unless clearly contradicted by context. Terms joined by or should be interpreted as inclusive, and terms joined by and should be interpreted as collective.

The description set forth herein provides a broad and flexible framework intended to support a wide range of computer-implemented, machine-learning-enabled, distributed, and multimodal embodiments. Variations may include reallocation of tasks, substitution of algorithms, reconfiguration of models, changes to pipeline ordering, or adoption of alternate hardware. No combination or arrangement mentioned herein should be regarded as required unless explicitly stated. The scope of protection is established by the claims, interpreted in light of this description.

The present disclosure contemplates implementations that employ advanced mathematical frameworks characteristic of modern artificial intelligence systems. Machine learning models may be conceptualized as parameterized functions that map elements of an input space to elements of an output space. Such a function may be defined over real-valued, complex-valued, vector-valued, tensor-valued, or mixed-modal domains. The model may implement successive transformations applied to an ordered set of input vectors using compositions of linear operators, nonlinear activations, attention functions, normalization operations, and dimensional projections.

Model parameters may be represented as ordered collections of real-valued scalars arranged into structures such as matrices, tensors, kernels, filters, or embeddings. These parameters may be optimized by minimizing a loss functional defined over an expected distribution of input-output pairs. The optimization process may involve computing gradients of the loss functional with respect to each model parameter, followed by an update step that serves to reduce the value of the loss functional. Gradient computation may use automatic differentiation frameworks that symbolically or numerically propagate partial derivatives backward through a computational graph.

Attention mechanisms may employ a similarity measure between projected query vectors and projected key vectors. This similarity measure may yield a weight distribution over contextual elements. The weighted combination of projected value vectors may form an attention output that is subsequently transformed through additional layers. Multiple independent attention heads may be aggregated to capture heterogeneous relationships within the input domain. Cross-attention mechanisms may operate similarly but with distinct source and target sequences.

Normalization steps may rescale intermediate representations using learned scaling and shifting coefficients. Activation functions may introduce nonlinearity by applying element-wise transformations selected to ensure differentiability and expressive capacity. Residual pathways may combine transformed and untransformed representations to facilitate stable gradient propagation under deep compositions. Positional encodings or structural embeddings may inject ordering, spatial, temporal, or relational information into otherwise permutation-invariant architectures.

Multi-modal models may operate over domains that combine text, image, audio, video, sensor, or structured signals. These domains may be embedded into a common vector space through learned projection operators. Joint training processes may enforce alignment constraints that minimize representational divergence between modalities while preserving intra-modal semantics.

Diffusion frameworks may model data generation as the reversal of a stochastic corruption process. A forward process may incrementally add noise to data samples, while a learned reverse process may approximate the time-reversed conditional probability distribution. The reverse process may be parameterized by a neural network trained to denoise intermediate states. Continuous-time formulations may model this process using stochastic differential equations whose drift and diffusion terms are learned through score-matching or related techniques.

Reinforcement-based procedures may model learning as an optimization of expected reward under a policy function. The policy may produce distributions over actions given a latent or explicit representation of the environment state. Policy gradients may be estimated from sampled trajectories, and advantage estimators may reduce the variance of such gradients. Value functions may approximate the expected cumulative reward, and these approximations may be updated through temporal-difference learning.

Generative models may be expressed in probabilistic terms as joint or conditional distributions parameterized by neural architectures. Such models may perform sampling by iteratively drawing latent variables from a learned distribution and transforming those variables into output space. Variational models may introduce auxiliary latent variables whose posterior distributions are approximated through recognition functions that optimize an evidence-bound objective.

Matrix decompositions, spectral analysis, manifold learning, kernel operators, and other mathematical constructs may be incorporated to improve expressiveness, stability, or computational efficiency. Training may involve sophisticated schedulers, trust-region constraints, adaptive learning-rate schemes, gradient-norm clipping, regularization penalties, entropy maximization, attention masking, or mixed-precision arithmetic.

All such mathematical constructs are conceptual, descriptive, and non-limiting. The disclosure encompasses any differentiable or non-differentiable optimization method, any discrete or continuous learning paradigm, and any representational transformation that may be understood by a person of ordinary skill in the field.

Further, the disclosure provides a computing environment which may include a combination of client devices, servers, distributed computing clusters, databases, external data sources, network nodes, and interface endpoints. Such an environment may support artificial intelligence workloads including perception, synthesis, inference, prediction, and generation, using hardware and software foundations designed for high-throughput and low-latency operation. Embodiments may involve the coordinated use of multiple machine learning models, whose configurations may evolve over time as they learn from training, validation, reference, or feedback data. Models may adjust their internal parameters through supervised, unsupervised, or reinforcement-based processes, allowing automatic electronic improvements to their performance based on input data and observed outcomes.

Further, the disclosure provides a computing device which may include processing units, memory elements, storage devices, system buses, high-speed controllers, low-speed controllers, and expansion interfaces. Processors may include general-purpose units, multi-core processors, vector processors, digital signal processors, tensor accelerators, neural accelerators, graphics engines, or various kinds of specialized integrated circuits including FPGAs, ASICs, ASSPs, SoCs, and CPLDs. A device may include system memory composed of volatile or non-volatile components such as RAM, DRAM, flash memory, ROM, or phase-change memory. The storage subsystem may include solid-state drives, magnetic disks, optical media, arrays of storage devices, and network-attached storage resources. Input and output mechanisms may include microphones, displays, keyboards, pointing devices, biometric sensors, gesture or touch interfaces, and actuators suitable for multimodal interaction with a user.

Further, the disclosure provides a machine-learning architecture which may include engines or modules such as a data input engine, data retrieval engine, data transform engine, featurization engine, modeling engine, generative engine, validation engine, feedback engine, and refinement engine. A data input pipeline may obtain structured or unstructured information from various sources, transform the information into model-compatible forms, and store such transformed data in memory or storage accessible to downstream components. A modeling engine may perform tasks such as model training, re-configuration, validation, and testing, executing iterative processes across multiple cycles or passes through training data. A predictive or generative engine may construct outputs based on intermediate representations, learned embeddings, or latent encodings generated by layers such as encoder-decoder structures, attention mechanisms, or multi-layer transformer architectures. Embeddings may represent discrete entities such as words, documents, or images as continuous vectors in high-dimensional spaces, capturing semantic or structural relationships useful for downstream tasks.

Further, the disclosure may provide a distributed or cloud-based operation may include multiple physical or virtual instances of computing devices, distributed across data centers or network boundaries. Functions may be partitioned across machines to achieve parallelism, redundancy, fault tolerance, or improved throughput. Distributed systems may use load balancing mechanisms to maintain stable processing, memory, or bandwidth utilization across clusters and avoid overload conditions. Such deployments may require communication over wired or wireless networks that implement a variety of protocols including HTTP, HTTPS, MQTT, CoAP, or any other suitable communication framework. Communication channels may include local networks, wide-area networks, personal-area networks, or global communication systems, potentially utilizing secure encrypted sessions such as SSL-based channels.

Further, the disclosure may provide an algorithm, process, or flow diagram which may include operations that may occur in sequences, reversed orders, concurrently, or in partially overlapping timelines, depending on the implementation. Blocks representing actions in a flowchart may correspond to program modules, instruction sequences, or hardware logic capable of performing the specified acts. Such operations may manipulate physical quantities such as electrical or magnetic signals stored or transferred among memory units, registers, storage devices, or communication media. Flow diagrams may be realized through software running on general-purpose processors, through dedicated hardware circuits, or through combinations of both.

Further, the disclosure may provide memory, storage, or programmatic constructs which may include program instructions encoded on computer-readable media including electronic, magnetic, optical, electromagnetic, semiconductor, or other tangible media. Examples include RAM, ROM, EEPROM, flash memory, magnetic disks, optical disks, and mechanical encoded structures such as punch cards or raised-pattern media. Such storage media may store instructions that, when executed, configure the memory and therefore configure the computing device itself, causing the device to perform functions described in association with the drawings.

Further, the disclosure may provide a user interface which may include graphical displays, dashboards, selection controls, input fields, monitoring elements, or multimodal interaction surfaces, allowing users to interact with computing systems in speech, touch, gesture, or other modalities. Such interfaces may be presented through client devices, server applications, or remote access platforms and may support visualization of model behavior, systems performance, or configuration parameters.

Further, described features may be combined, rearranged, omitted, or substituted without departing from the principles disclosed. Variations may involve distributing functionality across devices, merging components, implementing features in hardware rather than software, or employing alternative communication protocols. Many such variations and modifications are intended to fall within the scope of the disclosure as understood by persons skilled in the art.

The detailed description of the drawings therefore provides a foundation for describing technical, architectural, and operational aspects of embodiments, while allowing broad flexibility in how such embodiments may be implemented in practice. The scope of such embodiments is governed by the claims rather than the illustrative content of the drawings.

In some embodiments, a system consistent with this disclosure includes one or more client devices, one or more servers, and one or more data stores coupled by one or more networks. The client devices can include, without limitation, mobile phones, tablet computers, laptop or desktop computers, wearable devices, smart displays, vehicles, robots, or other computing platforms equipped with data processing hardware and memory hardware. The servers can include data servers, application servers, web servers, proxy servers, or cloud computing services that provide shared processing, storage, and networking resources. The data stores can include databases, object stores, file systems, or other repositories that persist configuration data, training data, logs, model artifacts, and other information.

The networks can include public and private networks, such as local area networks, wide area networks, and cloud networks, using wired or wireless communication links. The networks can provide routing, addressing, access control, encryption, and related functionality using standard or proprietary protocols.

Each computing device, whether a client device or a server, can include one or more processors, system memory, persistent storage, communication interfaces, and input or output devices. The processors can include general purpose central processing units, graphics processing units, digital signal processors, microcontrollers, application specific integrated circuits, field programmable gate arrays, or other programmable or fixed function processing elements configured to execute instructions or perform logic operations. The memory can include volatile and non-volatile storage, such as random access memory and read only memory. The persistent storage can include solid state drives, magnetic disks, optical media, or other non-transitory computer-readable media.

Program code executed by the processors can include operating systems, device drivers, libraries, and application programs, including components that implement portions of the methods described herein. Program code and data can be stored on computer-readable media and loaded into memory by standard mechanisms, such as boot loaders, installation programs, or update services.

Input devices can include keyboards, pointing devices, microphones, cameras, touch-sensitive surfaces, biometric sensors, and other sensors. Output devices can include displays, speakers, haptic devices, printers, and other actuators. Some devices can support multimodal interaction, allowing combined or sequential input and output through various modalities.

For purposes of this disclosure, artificial intelligence systems may include arrangements of software and hardware that perform tasks such as perception, prediction, planning, or generation based on input data. These systems can employ one or more models, such as statistical models, neural networks, decision trees, or other machine learning models. As used herein, a “model” can refer to a parameterized function, an ensemble of such functions, or a collection of cooperating components that process data and produce outputs.

In some embodiments, the system includes a data input engine that obtains data from one or more sources, such as application logs, sensor streams, structured databases, and unstructured content. The data input engine can retrieve, filter, aggregate, or transform the data into feature representations suitable for model consumption. Data sources can include training data, validation data, and reference data used to evaluate and calibrate model behavior.

A modeling engine can manage one or more training processes for one or more models. The modeling engine can select model architectures, initialize parameters, and apply training algorithms such as supervised learning, semi supervised learning, unsupervised learning, reinforcement learning, or combinations thereof. The modeling engine can also manage hyper parameters, training schedules, and evaluation procedures across epochs or passes through the data.

The system can include a generative response engine or inference engine that receives prompts or other inputs and generates outputs using one or more models. For example, a natural language interface can receive a text prompt, embed or otherwise encode the prompt, process the encoded prompt using a transformer based model or other sequence model, and generate a sequence of tokens that are decoded into an output. The engine can generate multiple candidate outputs and apply validation or ranking logic to select a final result according to quality, safety, or relevance criteria.

A feedback engine can collect explicit or implicit feedback signals, such as user ratings, corrective edits, or outcome metrics derived from downstream tasks. A refinement engine can use such feedback to adjust model parameters, routing logic, or policies, for example by performing additional training steps, updating reward models, or modifying configuration parameters.

In certain embodiments, the disclosed techniques are applied to platforms that include sensors and actuators, such as vehicles, robots, or other machines. The platform can include a processor system that receives signals from cameras, LIDAR units, radar units, inertial sensors, and other devices, and produces control outputs for steering, propulsion, braking, or other actuators. Sensor data can be captured at various sampling rates and processed by perception models to detect and track objects and infer scene attributes.

Planning and control components can receive outputs from perception models along with route information, traffic rules, and high-level goals. These components can generate trajectories or control commands, optionally using reinforcement learned policies, optimization-based planners, or hybrid systems. Connections to backend services can permit off-board processing, fleet-level learning, or remote supervision where appropriate, while on-board components can maintain safe operation in the presence of network latency or failures.

The systems described herein can be implemented using centralized, decentralized, or hybrid arrangements. For instance, models may be deployed in cloud environments, on edge devices, or across both, depending on requirements such as latency, privacy, cost, and reliability. Load balancing and resource management components can distribute processing across devices or data centers and can provide elasticity to accommodate changing workloads.

Certain embodiments may expose functionality through application programming interfaces, software development kits, or graphical user interfaces. Client applications can submit requests to backend services, which can apply authentication, authorization, logging, and policy enforcement before invoking models or tools and returning results.

The systems and methods disclosed herein can be implemented in hardware, software, firmware, or any combination thereof. In some embodiments, operations are carried out by one or more processors executing program instructions stored on one or more non-transitory computer-readable media. Such media can include, without limitation, semiconductor memory, magnetic storage, optical storage, and combinations thereof. Program instructions, when executed by the processors, cause the processors to perform the operations described herein.

Instructions can be delivered to computing devices in various ways, such as pre-installation, physical distribution of media, or transmission over networks. Instructions received over a network can be stored in memory or persistent storage and then executed by one or more processors. Dedicated hardware logic, such as application specific integrated circuits or field programmable gate arrays, can be used alone or in combination with software to implement certain functionality.

Any methods described in connection with embodiments of the present disclosure can be represented as one or more flow diagrams or state diagrams. Blocks in such diagrams can correspond to modules, components, operations, or code segments that implement the associated functionality. Blocks can be reordered, combined, executed concurrently, or omitted according to implementation specific considerations, unless a particular ordering is required by the claims.

Examples and embodiments described herein illustrate, rather than limit, the claimed subject matter. Certain features have been described in connection with particular embodiments for clarity, but other embodiments can include such features in different combinations. Features described in separate embodiments can be combined, and features described in a single embodiment can be separated, unless such combinations or separations are inconsistent with the claims. The scope of the disclosure is defined by the claims and their equivalents.

### Overview

The present disclosure relates to the use of temporal AI models for detecting situations and activities in sports and breaking them down into distinct phases. The methods and system disclosed herein may apply across various sports such as basketball, football, and hockey, enabling precise identification of key activities (e.g., shooting, passing, blocking) and detailed phase segmentation for performance analysis.

The methods and system may overcome that limitation by employing temporal AI models such as LSTM, GRU, RNN, and transformers, to analyze sports actions as sequences, detect situations and transitions, and segment them into phases. The use of 3D pose data may enrich the said models, allowing for detailed, real-time analysis of mechanics and activities under varying conditions, such as player fatigue or game context (e.g., guarded vs. unguarded shots).

Further, in some embodiments, the temporal AI model may include one or more of a multimodal model configured to process a video data and associated metadata; a vision-language model configured to perform prompt-conditioned classification; and a retrieval-augmented classification model for activity labels.

The present disclosure may provide a method for detecting sports situations and segmenting complex activities into phases using advanced temporal AI models. The integration of 3D pose data within the models ensures accurate phase segmentation and activity detection in real time. The present disclosure encompasses the following key features:
1. Situation Awareness Detection: Temporal models may identify sports-specific situations (e.g., on defense, on offense, shooting) and activities (e.g., jump shots, passes, blocks) from sequential data streams,
2. Activity Phase Segmentation: The system may break down activities into distinct phases (e.g., Transfer, Pocket, Release in basketball).
3. Adaptive Feedback Mechanism: The method and system may provide feedback in real time, and the feedback may be tailored to the detected situation, activity, and phase.
4. Integration of 3D Spatial Data Streams: The use of 3D data within the temporal models may enhance the precision of situation awareness and activity phase detection.
5. Flexible Model Architecture: The method and system may support LSTM, RNN, GRU, transformer models, or other temporal models, allowing for adaptability across different sports and use cases.

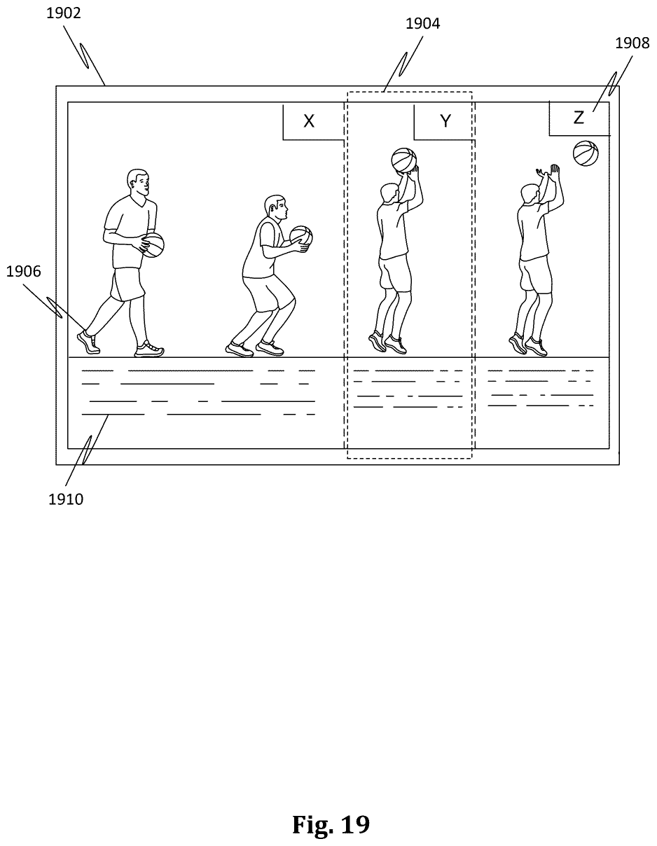

Further, the present disclosure provides the following detailed description of each key feature:
1. Situation Awareness Detection
The method and system use temporal AI models to detect situations and activities based on sequential data patterns. For example, in basketball, the system distinguishes between a jump shot, a free throw, and a layup. In football, the system identifies passes, tackles, or blocking activities. By analyzing sequential dependencies using models like LSTM, GRU, or transformers, the system recognizes complex actions in real time.
2. Activity Phase Segmentation and Analysis
Once a situation or event is detected, the system may segment the situation or event into phases for detailed analysis. For example, in basketball:
   Transfer Phase: Moving the ball into position for a shot.
   Pocket Phase: Aligning the ball with the shoulder for optimal release.
   Release Phase: Executing the shot with controlled wrist movement.
In football, phases may include pre-pass setup, release, and follow-through, or in hockey, stick preparation, puck release, and recovery. Each phase is evaluated against adaptive baselines, enabling athletes to monitor progress, make adjustments, and receive targeted feedback.
3. Integration of 3D Spatial Data
The method and system may integrate 3D pose data streams into the temporal models, allowing for spatially aware detection and phase segmentation, ensuring precise tracking of joint angles, movement patterns, and body alignment during performance analysis. For example, the system accounts for whether the player is guarded or unguarded, or if a football pass occurs under pressure from defenders. The spatial-temporal mechanics of a player playing Hockey is illustrated in FIG. 19.
4. Adaptive Feedback Mechanism
Real-time feedback is delivered across multiple platforms, including mobile apps, video overlays, and training systems. The feedback is customized to both the activity type and phase, such as correcting wrist angles during a basketball release phase or adjusting body posture during a football pass.
5. Flexible Model Architecture
The method and system accommodate various temporal AI models, including LSTM, GRU, RNN, transformer models, or other temporal models to analyze sequential data streams and deliver high-precision performance evaluations, ensuring adaptability across multiple sports and evolving AI architectures.

Further, the present disclosure encompasses the following aspects:
1. Method for Situation Awareness Detection:
   A method may detect sports-specific activities using temporal AI models, including LSTM, GRU, RNN, and transformers, to analyze sequential data streams.
2. System for Phase Segmentation:
   A system may segment detected activities into multiple phases and provide real-time feedback for each phase. Temporal AI models may be utilized for the said aspect as well.
3. Integration of Temporal Models with 3D Data:
   A method may integrate 3D spatial data streams into temporal AI models to enhance the accuracy of activity detection and phase segmentation.
4. Adaptive Feedback Mechanism:
   A feedback mechanism that provides real-time, situation-specific feedback based on the detected activity and phase.
5. Flexible Model Architecture:
   A system may be designed to support a variety of temporal AI models, ensuring adaptability across sports and evolving data environments.

Further, the method and system may analyze sports equipment (e.g., balls, hockey sticks, baseball bats, etc.) in addition to the biomechanical data for human motion. The sports equipment is as integral to athletic performance as the athlete's biomechanics, yet existing systems may not capture the said aspect. For instance:
Golf: While some systems visualize body key points, they fail to track and represent the club and the club's interaction with the swing plane.
Hockey: The stick's position and interaction with the puck are critical for shot accuracy and passing mechanics.
Basketball: Tracking both the ball's trajectory and player motion allows for comprehensive analysis of dribbling, shooting, and passing mechanics.

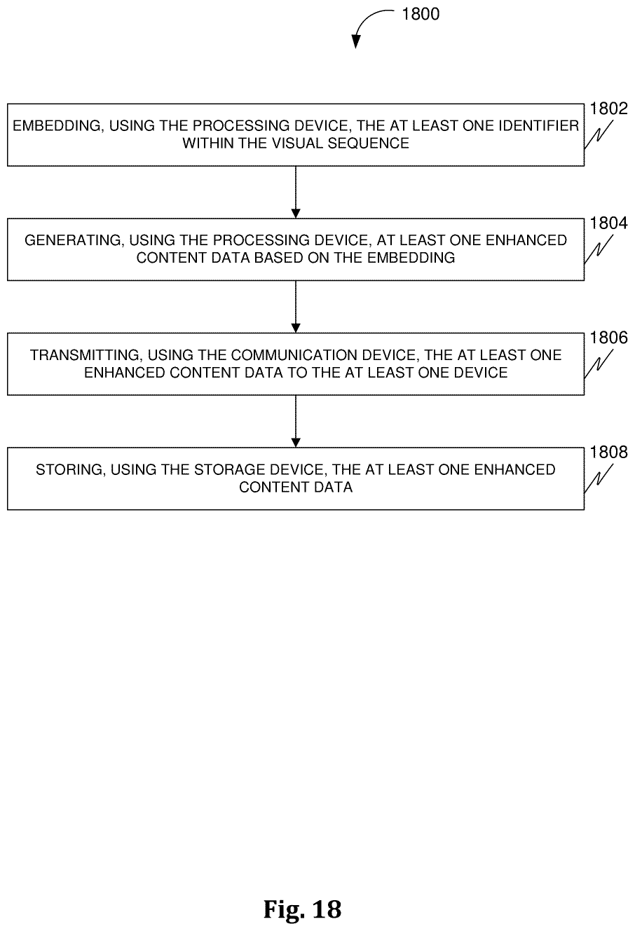

Further, the present disclosure provides a powerful system for situation awareness and phase detection in sports using advanced temporal AI models. By combining 3D data streams with sequential analysis, the system may deliver high-precision, real-time feedback tailored to the specific activity and phase. The real-time feedback tailored to the specific activity and phase is illustrated in FIG. 18. The flexibility of the architecture may ensure that the solution may adapt to a variety of sports, including basketball, football, and hockey, setting a new standard for real-time coaching and athlete improvement.

Further, the method disclosed herein may utilize artificial intelligence (AI) models, including but not limited to recurrent neural networks (RNNs), long short-term memory (LSTM) networks, or transformer-based models, to analyze temporal sequences of data associated with the physical activity. The said AI models may process sequential inputs such as sensor data, video frames, or other time-series data to identify patterns, predict outcomes, or generate metrics corresponding to the activity.

Further, the AI models may be configured to learn from historical data to improve accuracy and adaptability. For example, LSTM models may be used to capture long-term dependencies in time-series data, enabling the identification of complex biomechanical patterns, such as deviations in joint angles or movement trajectories, over time. Additionally, such models may provide real-time feedback or predictive insights by analyzing sequences of data as they are generated.

Further, the AI models may integrate with the disclosed method to determine deviations from reference metrics, identify key events, or provide actionable insights. Further, the disclosed method may include preprocessing steps such as feature extraction or dimensionality reduction to optimize data input for the AI models. The temporal AI model may include advanced hybrid architectures combining attention mechanisms, such as transformers, with recurrent layers like LSTMs to capture both local and global dependencies in sequential data.

Further, the system may support distributed processing across edge and cloud infrastructures, enabling real-time feedback generation for multiple users or scenarios simultaneously.
The system may include an explainability module that provides human-readable justifications for the feedback generated, such as highlighting key metrics or phases contributing to a performance score.
Handling Multimodal Data Streams:
The method and the system may analyze multimodal data streams, including synchronized video, audio, and motion sensor data, to enhance the contextual understanding of physical activities.
Real-Time Customization and Adaptability:
The feedback mechanism may dynamically adapt to real-time contextual variables such as athlete fatigue, weather conditions, or game strategy, ensuring tailored guidance.
Security and Data Privacy:
The system may incorporate privacy-preserving techniques, such as anonymized data processing and secure communication protocols, to ensure compliance with data protection regulations.
Use of Synthetic Data for Model Training:
The temporal AI model may be trained using synthetic data generated to simulate rare sports scenarios, such as unique biomechanical patterns or environmental conditions.
Hardware Optimization:
The system may include hardware-specific optimizations to ensure efficient performance on edge devices, such as smartphones or wearables, enabling real-time feedback even in resource-constrained environments.

Further, in some embodiments, the temporal AI model outputs an activity label corresponding to an activity instance represented in the content stream. Further, the temporal AI model outputs an activity confidence score, and wherein generating identifier data or feedback is gated based on the activity confidence score.

Further, in some embodiments, the activity label is selected from a plurality of sport activities.

Further, in some embodiments, the disclosed system facilitates detecting a start time and an end time for an activity instance within a continuous content stream. Further, the identifier data includes timestamps corresponding to the start time and end time.

Further, in some embodiments, the disclosed system facilitates determining a phase data for the activity instance, the phase data indicating a plurality of phases. Further, the disclosed system facilitates identifying a transition data representing a phase transition within temporal pattern data. Further, the phase transition is determined using at least one of: an attention-matrix signals, a hidden-state delta, or a velocity derivative of 3D pose landmarks. Further, the identifier data includes phase-based identifiers corresponding to the plurality of phases.

Further, in some embodiments, the situation data includes at least one contextual state selected from: a guarded vs an unguarded, a moving vs a still, posture state, a fatigue condition. Further, the disclosed system may facilitate determining of a fatigue metric and generating of a feedback based on the fatigue metric. Further, the disclosed system may facilitate determining of a situation condition data based on a physiological indicator data inferred from the situation data (fatigue model).

Further, in some embodiments, the present disclosure describes training the temporal AI model using historical activity data and storing a trained temporal AI model. Further, the temporal AI model is personalized using historical data associated with a particular athlete.

Further, in some embodiments, the present disclosure describes embedding at least one identifier within a visual sequence and generating enhanced content data based on the embedding. Further, the identifier data comprises metadata stored in association with the content stream data and accessible during playback for chaptering and retrieval.

Further, the receiving of a content stream data is based on a real-time communication protocol and feedback is transmitted based on the real-time communication protocol. Further, in some embodiments, the present disclosure may describe generating and transmitting a preliminary situation summary prior to final feedback generation (incremental/partial results).

Further, in some embodiments, the feedback data is anonymized using an anonymization technique prior to transmission.

Further, in some embodiments, the temporal AI model comprises at least one of: a recurrent neural network, a transformer model, a temporal convolutional network, a graph neural network, or a vision-language model. Further, the activity recognition is conditioned on contextual metadata or prompt text describing the sport activity. Further, the temporal AI model consumes multi-modal inputs including video and at least one of: IMU data, depth data, audio, or camera configuration metadata.

In some embodiments, the present disclosure relates to a computerized system and method for analyzing a content stream data corresponding to a physical activity using a temporal artificial intelligence model and generating a situation-based feedback. The disclosure inherently introduces several technical improvements to the functioning of computing systems, temporal data processing, streaming analytics, and model-driven feedback engines.

Further, the disclosed system introduces a set of inherent technical improvements that naturally arise from the architecture and operations of the disclosed system. The said improvements enhance the operation of temporal AI processing, real-time content stream analysis, multimodal data fusion, and contextual feedback generation.

In some embodiments, the system may provide an improved temporal sequence analysis capability in which a temporal AI model may be used to process high-dimensional sequential data in a manner that enhances the accuracy, speed, and contextual relevance of physical-activity interpretation. The technical problem addressed by the given improvement arises from the inability of conventional static models or frame-based recognition systems to capture long-range dependencies in physical motion. Such conventional approaches may fail to detect subtle differences between phases of an athletic movement or may misinterpret temporally interdependent sequences. The disclosed system may solve the given problem by applying a recurrent or transformer-based temporal model trained specifically on spatial-temporal sequences. The system may incorporate long short-term memory networks that model dependencies across varied time windows, gated recurrent units that capture transitional movement shifts with low computational cost, or transformer models that compute attention weights across non-adjacent frames to interpret complex motion. The given improvement enhances the specific technology of temporal sequence modeling and sequence-to-label machine learning pipelines by enabling the model to capture biomechanical nuances that are otherwise lost.

In some embodiments, the system may include an improved real-time content stream processing architecture that maintains high throughput while performing advanced temporal inference. The technical problem addressed by the given improvement involves latency accumulation resulting from computationally heavy AI inference performed on continuous video or sensor streams. Legacy systems either drop frames, produce inconsistent analysis, or require offline batch processing. The disclosed system may solve the given issue by segmenting the stream into micro-sequences, generating an intermediate situation data in near real time, and queuing subsequent data for pipelined inference. The system may additionally employ partial inference caching or edge-cloud cooperative processing to reduce server congestion. The given improvement directly enhances streaming analytics technology and enables real-time interpretation of high-volume sequential data.

In some embodiments, the system may include an improved multimodal spatial-temporal data fusion capability. The technical problem addressed concerns the difficulty of combining raw sensor data, video frames, annotated pose data, and time-series metrics into a coherent representation that may be interpreted by a temporal AI model. Traditional fusion architectures may exhibit misalignment problems, timing drift, or feature-space mismatches across modalities. The system may address the said limitations by establishing a synchronized sequential identifier data embedded with timestamp data and temporal offset information. The system may generate a unified vectorized representation that incorporates three-dimensional spatial data, joint-angle data, body-alignment data, and sport-object trajectory data. In some embodiments, the system may use time-warping or Kalman-filter smoothing to align modalities prior to temporal analysis. The given improvement enhances multimodal machine learning technology by enabling the fusion of diverse spatial-temporal sources in a real-time environment.

In some embodiments, the system may provide an improved situation-aware activity decomposition capability in which the temporal AI model may break an activity into phases based on temporal cues discovered within the content stream data. The technical problem addressed concerns the difficulty of segmenting athletic movements into bounded phases, such as transfer, pocket, release, or follow-through, because such phases lack clear visual boundaries in raw data. The system may solve the said issue using transition-point detection generated from internal attention matrices, hidden state deltas, or velocity derivatives of three-dimensional pose landmarks. The model may compute transitional micro-patterns that delineate phase boundaries even under occlusion, varying camera angles, or inconsistent lighting, ensuring the technology of activity phase segmentation and biomechanical decomposition.

In some embodiments, the system may provide an improved feedback-generation mechanism in which contextually meaningful feedback is derived from situation data rather than from generic threshold-based rules. The technical problem addressed arises from existing athletic feedback systems relying on static metrics without considering situation conditions such as fatigue, defensive pressure, or environmental variations. The disclosed system may compute situation-dependent metrics by comparing extracted biomechanical characteristics with a dynamic reference metric selected based on the current detected situation. The said metrics may include separate baselines for guarded versus unguarded activity, pressured versus relaxed conditions, or fatigued versus unfatigued motion profiles, ensuring feedback-generation technology by transforming the process from static rule engines into dynamic contextual evaluators.

In some embodiments, the system may include an improved representation of sport-object characteristics derived from athlete biomechanics. The technical problem addressed is that conventional systems treat sport objects (such as a ball, bat, club, or stick) as independent entities requiring separate sensors or object-specific tracking models. The disclosed system may infer sport object mechanics, such as angle, trajectory, or alignment, based solely on athlete kinematics using the temporal AI model. For example, the system may infer the orientation of a hockey stick from wrist-boarding angle and shoulder pose, or may estimate a basketball's release trajectory based on acceleration and orientation of the hand, directly improving the technology of object-motion inference within biomechanical modeling.

In some embodiments, the system may include an improved sequential-dependency interpretation mechanism. The technical problem addressed involves the inability of traditional movement-analysis systems to link prior activity segments to subsequent phases of performance, causing incomplete or incorrect performance analysis. The invention may compute sequential dependencies by examining sequential identifier data, identifying ordering rules, and deriving transition probabilities between events. For example, the system may discover that a suboptimal pocket phase consistently precedes a delayed release phase. The given improvement enhances temporal-dependency modeling as applied to athletic biomechanics.

In some embodiments, the system may incorporate an improved fatigue inference engine. The technical problem addressed concerns real-time detection of athlete fatigue without requiring special-purpose biomonitoring sensors. The invention may infer fatigue based on spatial-temporal mechanics, such as reduced joint extension velocity, altered posture, prolonged transition times, or inconsistent phase timing. A fatigue model data may be trained on annotated fatigue examples and may use characteristic deviations from baseline mechanics to produce a situation condition data, improving computational physiological-state estimation technology.

In some embodiments, the system may provide an improved real-time communication protocol handling for delivering incremental situation data before full feedback computation. The technical problem addressed involves the need to deliver early warnings or preliminary analysis to a remote coach or athlete before the entire analysis pipeline completes. The system may prepare a preliminary situation summary data derived from partial inference, store it temporarily, and transmit the preliminary situation summary data ahead of the final feedback data, ensuring real-time interactive analytics technology, particularly for cloud-based performance monitoring systems.

In some embodiments, the system may include an adaptive temporal-resolution mechanism that dynamically adjusts the frame or sample rate of the content stream data based on real-time model uncertainty. The technical problem addressed concerns inefficient resource allocation when analyzing data segments that exhibit low temporal complexity. The system may increase the sampling density during rapid transitions (such as release moments) and may reduce the sampling density during periods of minimal motion. The given enhancement may improve temporal-sampling technology and reduce computational overhead in real-time streaming environments.

In some embodiments, the system may include a self-supervised representation learning module that pre-trains the temporal AI model on unlabeled motion sequences to reduce annotation requirements. The technical problem addressed is that manual labeling of phases, activities, and biomechanical factors is costly and error-prone. The system may employ contrastive sequence learning, masked motion reconstruction, or temporal-order prediction tasks to pre-train the model. The given improvement may enhance the technology of AI pre-training for biomechanical intelligence systems.

In some embodiments, the system may include a dynamic reference-metric generation engine that constructs personalized baseline metrics using federated learning. The technical problem addressed involves generating accurate baseline metrics without centralizing sensitive performance data from multiple athletes. The system may use federated gradient aggregation to update a shared baseline model while keeping athlete-specific data decentralized, improving privacy-preserving model-training technology.

In some embodiments, the system may incorporate an edge-acceleration layer that performs pre-processing operations directly on a content source device or nearby edge node. The technical problem addressed concerns bandwidth limitations and redundant server-side computations. The system may perform pose extraction, frame normalization, or delta-compression at the network edge to reduce upload overhead and accelerate server-side inference, enhancing the technology of distributed AI inference pipelines.

In some embodiments, the system may incorporate an explainability module configured to generate interpretable biomechanical indicators or visual cues that explain the temporal AI model's decisions. The technical problem addressed involves the opacity of deep temporal models, which limits user trust and inhibits training effectiveness. The system may generate feature-importance maps, phase-transition saliency curves, or biomechanical deviation markers derived from the situation data, which may be transmitted in a structured form, improving explainable-AI technology applied to real-time athletic analysis.

Further, the present disclosure describes a method for facilitating situation awareness for a physical activity.

Further, in some embodiments, the method may include receiving, using the communication device, the at least one content data from the at least one content source device. Further, the at least one content source device is and/or comprises a camera and an inertial measurement unit (IMU) sensor. Further, the at least one content data comprises video data captured by the camera and motion data captured by the IMU sensor. Further, the at least one content data may be organized into portions. Further, a portion comprises at least one video frame, a set of consecutive video frames, and a corresponding time-aligned set of IMU samples.

Further, in some embodiments, the method may include determining, using the processing device, at least one entity region corresponding to at least one entity and at least one background region corresponding to a background of the at least one entity from the at least one content data. Further, the determining of the at least one entity region and the at least one background region may include generating at least one of a region-of-interest (ROI) mask and a segmentation mask from the video data, wherein at least one of the ROI mask and the segmentation mask delineates pixels corresponding to the at least one entity and pixels corresponding to the background. Further, at least one of the ROI mask and the segmentation mask may be produced using at least one of a trained detector model and a segmentation model stored in a memory and executed by the processing device. Further, at least one of the detector model and the segmentation model outputs at least one bounding box, at least one mask, and at least one confidence value.

Further, in some embodiments, the method may include extracting, using the processing device, a plurality of entity feature vectors from the at least one entity region and a plurality of background feature vectors from the at least one background region. Further, the plurality of entity feature vectors may comprise at least one of appearance features, pose features, motion features, or fused video-IMU features derived from the at least one content data. Further, the plurality of background feature vectors may comprise at least one of scene texture features, illumination features, camera motion features, or encoder artifact features derived from the at least one content data. Further, each entity feature vector may be paired with a corresponding background feature vector derived from a same portion of the at least one content data, such that the pairing associates contemporaneous entity and background information from the same time interval.

Further, in some embodiments, the method may include computing, using the processing device, for each paired entity feature vector and corresponding background feature vector, at least one contextual-correlation value that indicates whether the paired features (i.e., paired entity feature vector and corresponding background feature vector) represent background-induced interference with respect to the at least one entity. Further, the at least one contextual-correlation value may be computed as a bounded value indicating a degree of similarity or dependency between the paired entity feature vector and the corresponding background feature vector. Further, the computing of the contextual-correlation value may include computing at least one similarity metric between the paired vectors, including a cosine similarity, a normalized correlation, or an attention-derived similarity, and may further include incorporating at least one IMU-derived motion component indicating camera shake, abrupt rotation, or vibration that contributes to background-induced interference. Further, background-induced interference may include at least one of motion blur, camera shake, rapid background motion, illumination fluctuation, or compression artifacts that reduce separability between entity features and background features.

Further, in some embodiments, the method may include filtering, using the processing device, the paired entity feature vectors and corresponding background feature vectors based on the contextual-correlation value relative to at least one threshold to generate a reduced set comprising a reduced plurality of entity feature vectors and a corresponding reduced plurality of background feature vectors. Further, the threshold may be predetermined, learned, or adaptively selected based on at least one statistic of the contextual-correlation values computed over a plurality of portions of the at least one content data. Further, the filtering may include excluding, masking, or down-weighting paired features that are determined to be affected by background-induced interference based on the contextual-correlation value, and retaining paired features that satisfy an interference criterion.

Further, in some embodiments, the method may include selectively bypassing, using the processing device, feature propagation for at least one paired feature excluded by the filtering, wherein the selectively bypassing comprises implementing compute gating in the at least one temporal AI model. Further, the compute gating may include generating a gating mask corresponding to excluded paired features and applying the gating mask to at least one intermediate tensor of the at least one temporal AI model, such that at least one layer operation is skipped for masked elements (i.e., excluded paired features). Further, the selectively bypassing may include bypassing at least one multiply-accumulate operation, bypassing at least one attention computation, bypassing at least one matrix multiplication, or performing sparse inference on masked activations, such that a number of processor cycles and a memory bandwidth consumed during inference are reduced. Further, the compute gating may be performed to satisfy at least one real-time constraint for facilitating situation awareness for the physical activity, including maintaining inference latency below a predetermined latency budget and maintaining power consumption below a predetermined power budget on a mobile or wearable implementation.

Further, in some embodiments, the method may include identifying, using the processing device, the at least one situation based on the reduced set using the at least one temporal AI model, wherein the at least one temporal AI model comprises at least one temporal model configured to process time-ordered inputs, including a temporal convolution model, a recurrent model, or a transformer-based temporal model. Further, the identifying of the at least one situation based on the reduced set may improve robustness of situation identification under background-induced interference and may reduce computational complexity by avoiding propagation of masked features through the temporal model, thereby improving real-time performance for the physical activity. Further, the identifying of the at least one situation may include the identifying of the situation data.

Further, in some embodiments, the method may include determining, using the processing device, at least one technical condition associated with the at least one content data that contributes to background-induced interference with respect to identifying the at least one situation. Further, the at least one technical condition may include at least one of motion blur, low illumination, sensor noise, occlusion, camera shake, compression artifacts, or packet loss. Further, the determining of the at least one technical condition may include computing at least one measurable metric from the video data and the motion data, including a blur metric derived from image gradients, a luminance metric derived from pixel intensity statistics, a noise metric derived from a signal-to-noise estimate, a camera shake metric derived from the IMU samples, and an artifact metric derived from encoded video statistics.

Further, in some embodiments, the method may include generating, using the processing device, at least one control data based on at least one of (i) the contextual-correlation value, (ii) a quantity or proportion of paired features excluded by the filtering, and (iii) the at least one technical condition. Further, the control data may specify at least one device parameter for the at least one content source device, wherein the at least one device parameter comprises at least one of an exposure setting, a shutter setting, a sensor gain setting, a frame rate setting, an image stabilization setting, an encoder bitrate setting, an encoder quantization setting, or a sampling rate setting of the IMU sensor and/or the camera. Further, the method may include transmitting, using the communication device, the at least one control data to the at least one content source device, and configuring, using the at least one content source device, the at least one device parameter based on the control data such that at least one subsequent content data is captured or encoded with reduced background-induced interference for identifying the at least one situation.

Further, in some embodiments, the method may include configuring, using the at least one content source device, an image stabilization setting or a frame rate setting responsive to determining that camera shake exceeds a threshold based on the motion data. Further, the method may include configuring, using the at least one content source device, an exposure setting and a sensor gain setting responsive to determining that motion blur or low illumination contributes to background-induced interference. Further, the method may include configuring, using the at least one content source device, an encoder bitrate setting or an encoder quantization setting responsive to determining that compression artifacts contribute to background-induced interference. Further, the method may include configuring, using the at least one content source device, an IMU sampling rate setting responsive to determining that improved motion resolution is required for time-alignment between the motion data and the video data.

Further, in some embodiments, the method may include receiving, using the communication device, the at least one subsequent content data from the at least one content source device after configuring the at least one device parameter, and identifying, using the processing device, the situation data using the at least one temporal AI model based on the at least one subsequent content data.

Further, in some embodiments, the term “portion” refers to a time-bounded segment of the at least one content data comprising at least one video frame and a corresponding set of IMU samples that overlap the time of the at least one video frame. Further, the term “paired” refers to associating an entity feature vector and a background feature vector derived from the same portion. Further, the term “contextual-correlation value” refers to a numeric value computed according to a predetermined correlation function between the paired vectors and compared to a threshold to determine whether the paired features are excluded from the reduced set. Further, the term “background-induced interference” refers to measurable conditions in the at least one content data that reduce separability of entity features from background features and degrade identification of the at least one situation, including motion blur, camera shake, and compression artifacts.

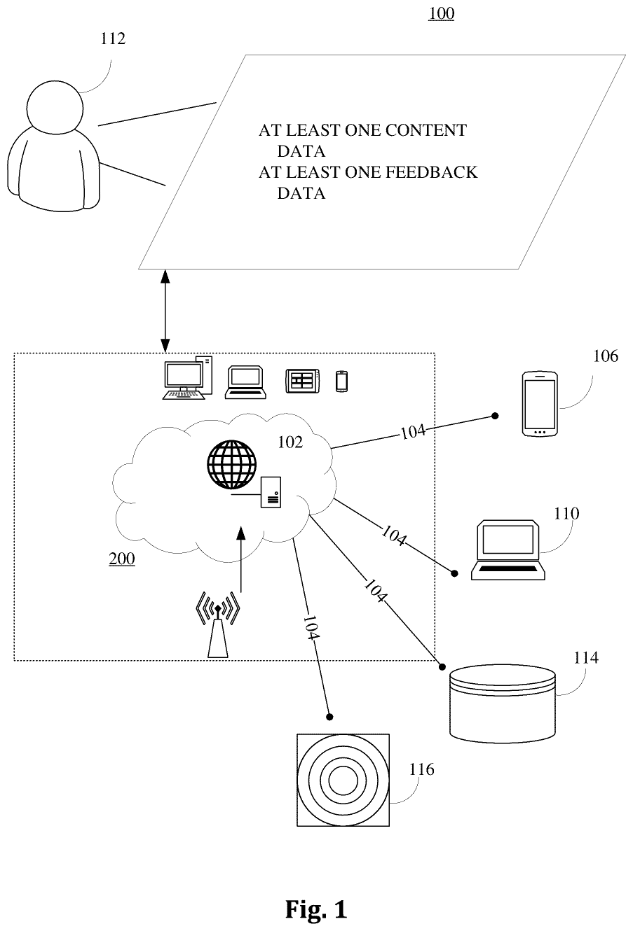

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

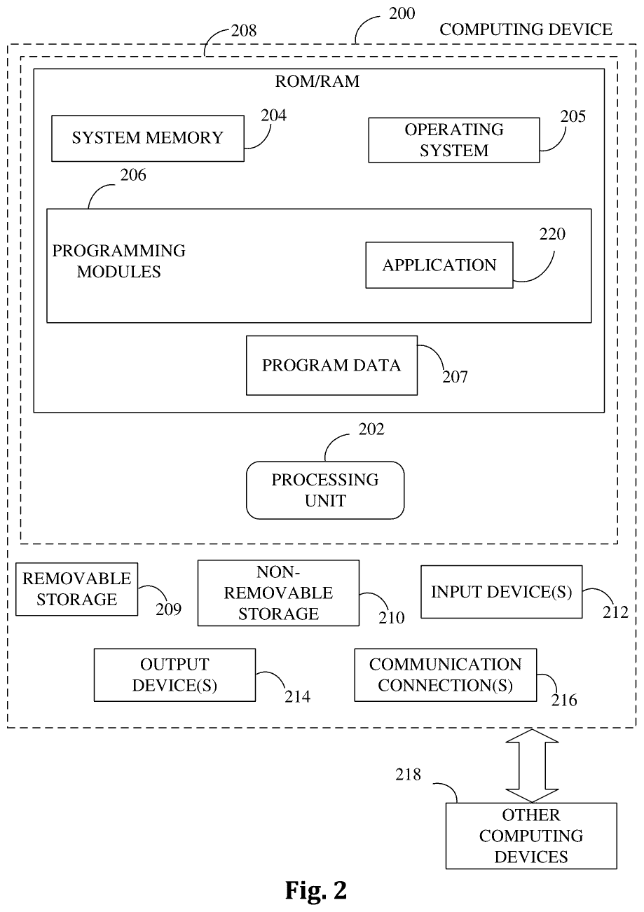

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods'stages may be modified in any manner, including by reordering stages and/or inserting or deleting stages, without departing from the disclosure.

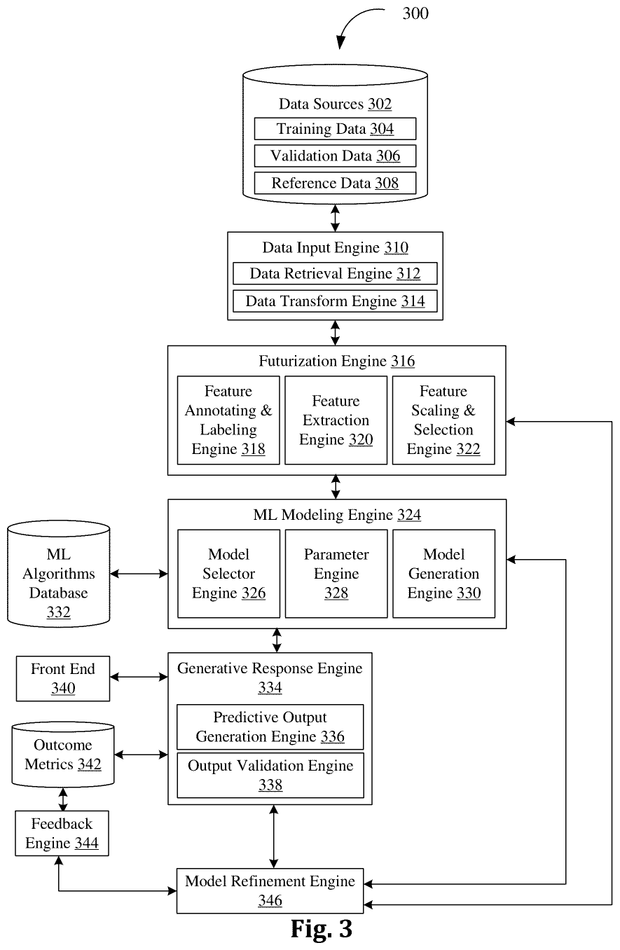

FIG. 3 is a block diagram illustrating a machine-learning system 300 for implementing various embodiments of this disclosure, in accordance with some embodiments. Although the disclosed machine-learning system 300 depicts particular system components and an arrangement of such components, the given depiction is to facilitate a discussion of the present technology and should not be considered limiting unless specified in the appended claims. For, example some components that are illustrated as separate, may be combined with other components and some components may be divided into separate components.

Accordingly, the machine-learning system 300 may include a plurality of interrelated modules and engines configured to implement a machine-learning pipeline. Further, the machine-learning system 300 may include a data sources module 302 that is made up of a training data repository 304, a validation data repository 306, and a reference data repository 308, each repository being configured to store respective classes of input records and reference information. Further, the machine-learning system 300 may include a data input engine 310 configured to receive data from the data sources module 302. Further, the data input engine 310 may include a data retrieval engine 312 configured to access and ingest data from the repositories (304, 306, 308), and a data transform engine 314 configured to perform initial normalization, parsing and format conversion on the ingested data. Further, the machine-learning system 300 may include a featurization engine 316 configured to prepare temporal and predictive representations of transformed data. Further, the featurization engine 316 may include a feature annotating & labeling engine 318 for applying labels and annotations to data instances, a feature extraction engine 320 for deriving feature vectors and candidate predictors, and a feature scaling & selection engine 322 for performing numerical scaling, dimensionality reduction and selection of salient features. Further, the machine-learning system 300 may include a machine learning (ML) modeling engine 324 configured to construct predictive models from selected features. Further, the ML modeling engine 324 may include a model selector engine 326 for selecting among candidate model classes, a parameter engine 328 for determining and tuning hyper parameters, and a model generation engine 330 for instantiating and training model artifacts according to selected architectures and parameters. Further, the machine-learning system 300 may include an ML algorithms database 332 configured to store algorithmic implementations, model templates and associated metadata and to be accessible by components of the ML modeling engine 324. Further, the machine-learning system 300 may include a generative response engine 334 configured to produce user-facing outputs based on the trained models. Further, the generative response engine 334 may include a predictive output generation engine 336 for generating predictions or synthesized responses and an output validation engine 338 for verifying, filtering and validating generated outputs against predefined criteria and reference data. Further, the machine-learning system 300 may include a front end 340 configured to present validated outputs to end users and to collect interaction signals. Further, the machine-learning system 300 may include an outcome metrics module 342 configured to compute performance measures, accuracy statistics and other evaluation metrics derived from model outputs and user interactions. Further, the machine-learning system 300 may include a feedback engine 344 configured to aggregate outcome metrics and user feedback and to format such information for reuse. Further, the machine-learning system 300 may include a model refinement engine 346 configured to receive feedback from the feedback engine 344 and the outcome metrics module 342, and to effect iterative updates to the ML modeling engine 324 and to the ML algorithms database 332. Further, the components are communicatively coupled so that data and control signals are exchanged among the repositories (304, 306, 308), the data input engine 310, the featurization engine 316, the ML modeling engine 324 (with algorithmic support from the ML algorithms database 332), the generative response engine 334 and the front end 340 for output generation. Further, the outcome metrics 342 and feedback engine 344 provide closed-loop signals to the model refinement engine 346 to enable retraining, parameter adjustment and algorithm selection, thereby enabling cooperative execution of data acquisition, feature engineering, model construction, output generation, validation, evaluation and iterative refinement within the disclosed machine-learning system 300.

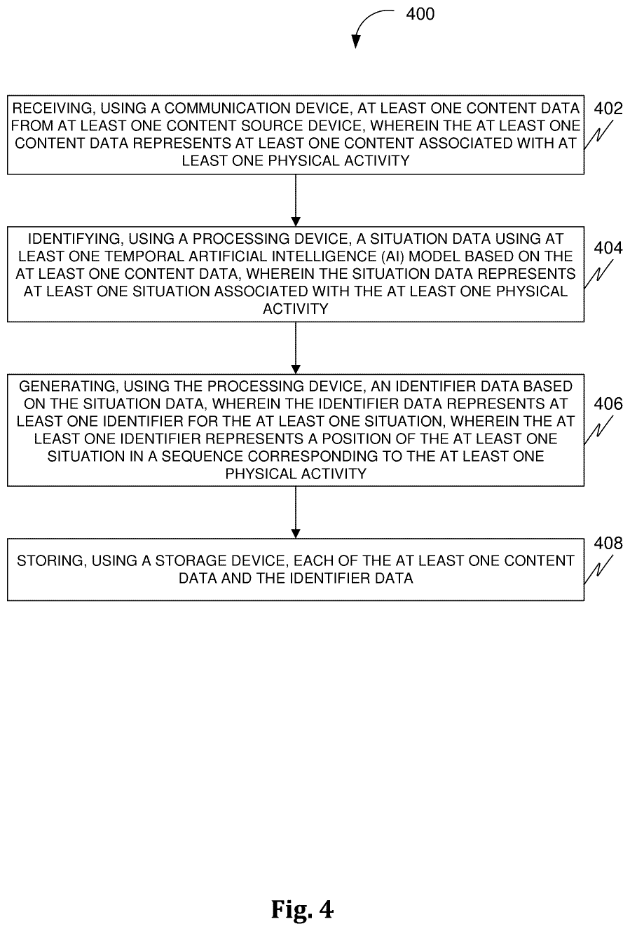

FIG. 4 illustrates a flowchart of a method 400 for facilitating situation awareness for a physical activity, in accordance with some embodiments. Accordingly, the method 400 may include a step 402 of receiving, using a communication device 1002, one or more content data from one or more content source devices. Further, the one or more content data represent one or more contents associated with one or more physical activities. Further, the method 400 may include a step 404 of identifying, using a processing device 1004, a situation data using one or more temporal artificial intelligence (AI) models based on the one or more content data. Further, the situation may data represent one or more situations associated with the one or more physical activities. Further, the method 400 may include a step 406 of generating, using the processing device 1004, an identifier data based on the situation data. Further, the identifier data represents one or more identifiers for the one or more situations. Further, the one or more identifiers represent a position of the one or more situations in a sequence corresponding to the one or more physical activities. Further, the method 400 may include a step 408 of storing, using a storage device 1006, each of the one or more content data and the identifier data.

Further, in some embodiments, the identifying of the situation data may include identifying an event data representing one or more events associated with the one or more physical activities using the one or more temporal AI models. Further, the identifying of the situation data may include identifying a phase data representing one or more phases associated with the one or more events. Further, the one or more identifiers include one or more phase-based identifiers. Further, the generating of the identifier data includes generating a phase-based identifier data based on the identifying of the phase data. Further, the phase-based identifier data represents the one or more phase-based identifiers associated with the one or more physical activities.

In some embodiments, the identifier data includes one or more of a time stamp data representing a time value associated with the one or more situations and a time interval data representing a time interval associated with the one or more situations relative to the one or more contents.

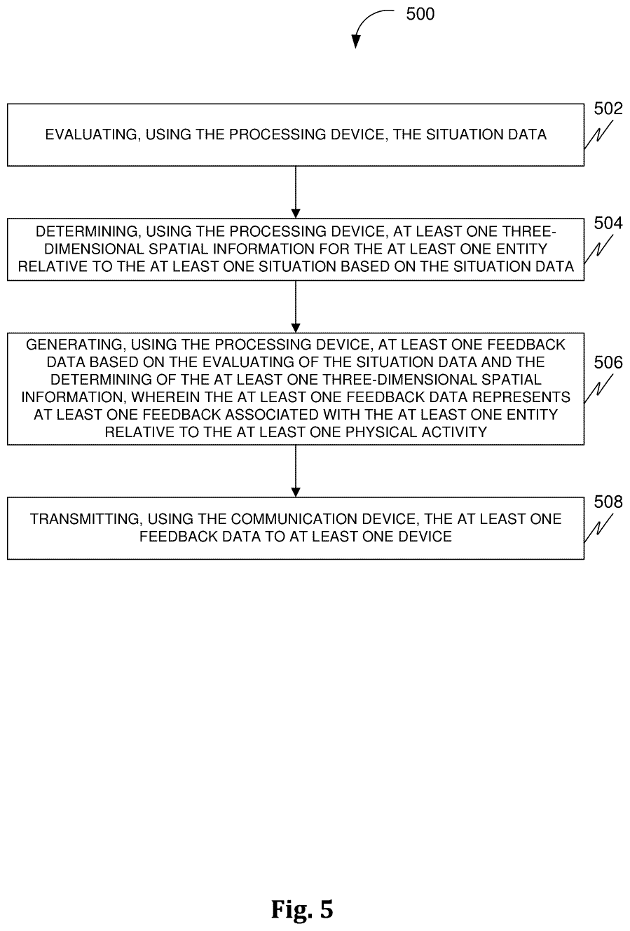

FIG. 5 illustrates a flowchart of a method 500 for facilitating situation awareness for a physical activity including generating, using the processing device 1004, at least one feedback data, in accordance with some embodiments. Further, in some embodiments, the one or more physical activities may be associated with one or more states of one or more entities. Further, the method 500 further may include a step 502 of evaluating, using the processing device 1004, the situation data. Further, the method 500 further may include a step 504 of determining, using the processing device 1004, one or more three-dimensional spatial information for the one or more entities relative to the one or more situations based on the situation data. Further, the method 500 further may include a step 506 of generating, using the processing device 1004, one or more feedback data based on the evaluating of the situation data and the determining of the one or more three-dimensional spatial information. Further, the one or more feedback data represents one or more feedback associated with the one or more entities relative to the one or more physical activities. Further, the method 500 further may include a step 508 of transmitting, using the communication device 1002, the one or more feedback data to one or more devices 1008.

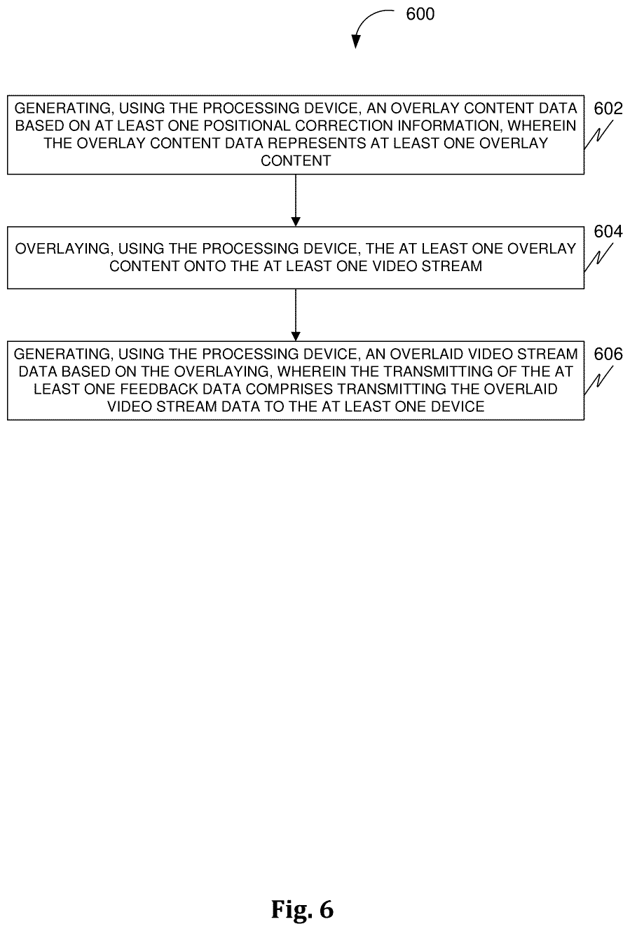

FIG. 6 illustrates a flowchart of a method 600 for facilitating situation awareness for a physical activity including generating, using the processing device 1004, an overlaid video stream data, in accordance with some embodiments. Further, in some embodiments, the one or more content data may include a video stream data representing one or more video streams associated with the one or more physical activities. Further, the one or more feedbacks may include one or more positional correction information associated with the one or more entities relative to the one or more physical activities. Further, the method 600 further may include a step 602 of generating, using the processing device 1004, an overlay content data based on one or more positional correction information. Further, the overlay content data represents one or more overlay contents. Further, the one or more content data may include a video stream data representing one or more video streams associated with the one or more physical activities. Further, the method 600 further may include a step 604 of overlaying, using the processing device 1004, the one or more overlay contents onto the one or more video streams. Further, the one or more content data may include a video stream data representing one or more video streams associated with the one or more physical activities. Further, the method 600 further may include a step 606 of generating, using the processing device 1004, an overlaid video stream data based on the overlaying. Further, the transmitting of the one or more feedback data includes transmitting the overlaid video stream data to the one or more devices 1008.

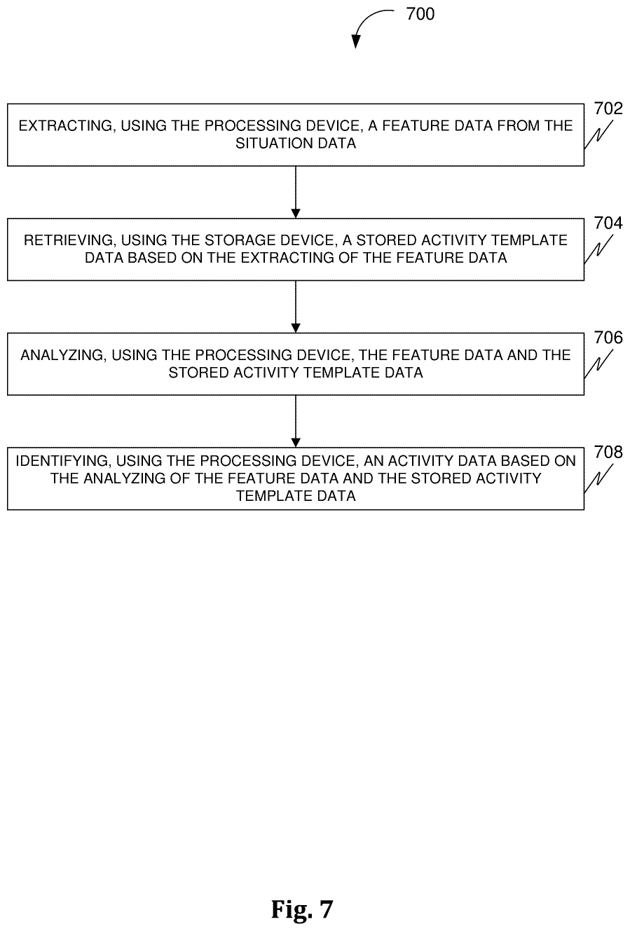

FIG. 7 illustrates a flowchart of a method 700 for facilitating situation awareness for a physical activity including identifying, using the processing device 1004, an activity data, in accordance with some embodiments. Further, in some embodiments, the method 700 further may include a step 702 of extracting, using the processing device 1004, a feature data from the situation data. Further, in some embodiments, the method 700 further may include a step 704 of retrieving, using the storage device 1006, a stored activity template data based on the extracting of the feature data. Further, in some embodiments, the method 700 further may include a step 706 of analyzing, using the processing device 1004, the feature data and the stored activity template data. Further, in some embodiments, the method 700 further may include a step 708 of identifying, using the processing device 1004, an activity data based on the analyzing of the feature data and the stored activity template data.

Further, the activity data corresponds to the one or more physical activities. Further, the activity data may include an activity-specific boundary detection data representing at least one boundary detection parameter relative to the one or more physical activities. Further, the method 400 may further include a step of evaluating, using the processing device 1004, the activity-specific boundary detection data using the one or more temporal AI models. Further, the identifying of the situation data is further based on the evaluating of the activity-specific boundary detection data.

In some embodiments, the one or more devices 1008 include one or more coach devices 1102 associated with one or more coaches. Further, the transmitting of the one or more feedback data to the one or more devices 1008 includes transmitting the one or more feedback data to the one or more coach devices 1102. Further, the one or more coach devices 1102 include a coach-side presentation device which may be configured for presenting the one or more feedback to the one or more coaches. Further, the one or more coach devices 1102 further include a coach-side input device which may be configured for generating an input data representing one or more inputs relative to the one or more feedbacks. Further, the one or more coach devices 1102 further include a coach-side communication device, which may be configured for transmitting the input data to the communication device 1002.

In some embodiments, the one or more content data includes one or more sensor data associated with one or more sensor devices, which may be configured for monitoring the one or more physical activities. Further, the method 500 further includes analyzing, using the processing device 1004, the one or more sensor data. Further, the determining of the one or more three-dimensional spatial information for the one or more entities may be further based on the analyzing of the one or more sensor data.

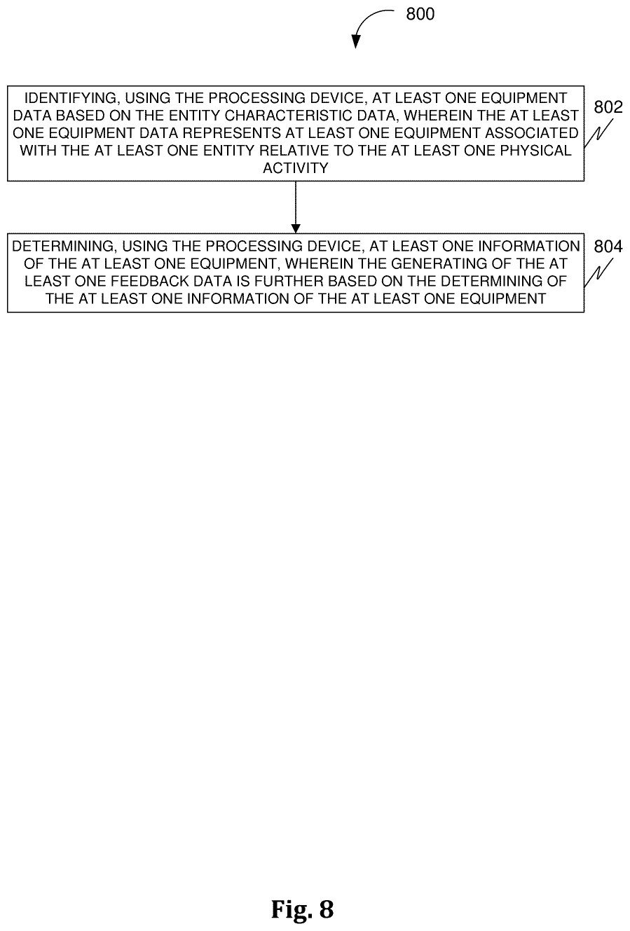

FIG. 8 illustrates a flowchart of a method 800 for facilitating situation awareness for a physical activity including determining, using the processing device 1004, at least one information of the at least one equipment, in accordance with some embodiments. Further, in some embodiments, the method 800 further may include a step 802 of identifying, using the processing device 1004, one or more equipment data based on the situation data. Further, the one or more equipment data represents one or more equipment associated with the one or more physical activities. Further, in some embodiments, the method 800 further may include a step 804 of determining, using the processing device 1004, one or more information of the one or more equipment. Further, the generating of the one or more feedback data may be further based on the determining of the one or more information of the one or more equipment.

In some embodiments, the identifier data includes one or more of a time stamp data representing a time value associated with the one or more situations and a time interval data representing a time interval associated with the one or more situations relative to the one or more contents.

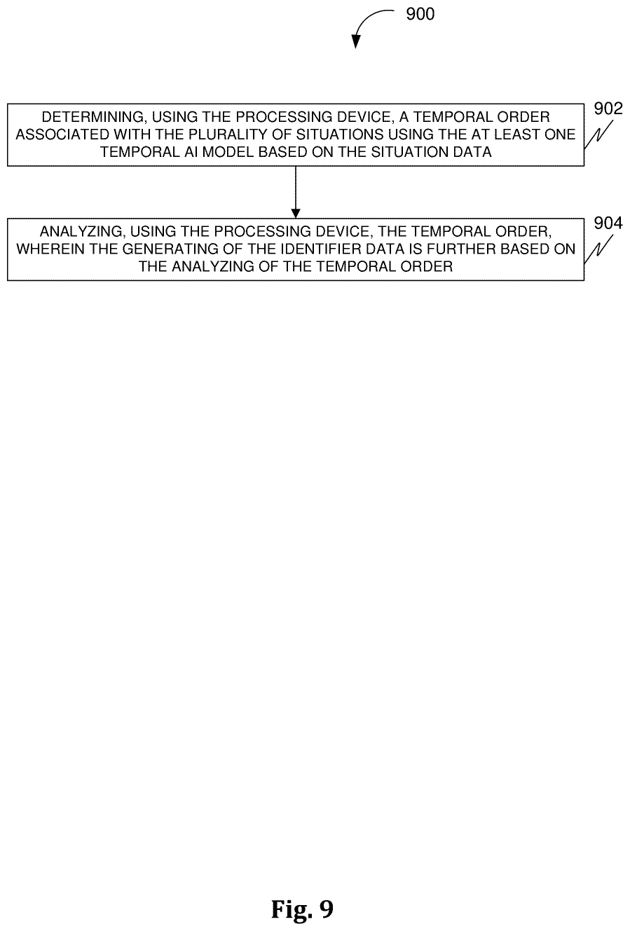

FIG. 9 illustrates a flowchart of a method 900 for facilitating situation awareness for a physical activity including analyzing, using the processing device 1004, the temporal order, in accordance with some embodiments. Further, in some embodiments, the one or more situations may include two or more situations. Further, the method 900 further may include a step 902 of determining, using the processing device 1004, a temporal order associated with the two or more situations using the one or more temporal AI models based on the situation data. Further, the method 900 further may include a step 904 of analyzing, using the processing device 1004, the temporal order. Further, the generating of the identifier data may be further based on the analyzing of the temporal order.

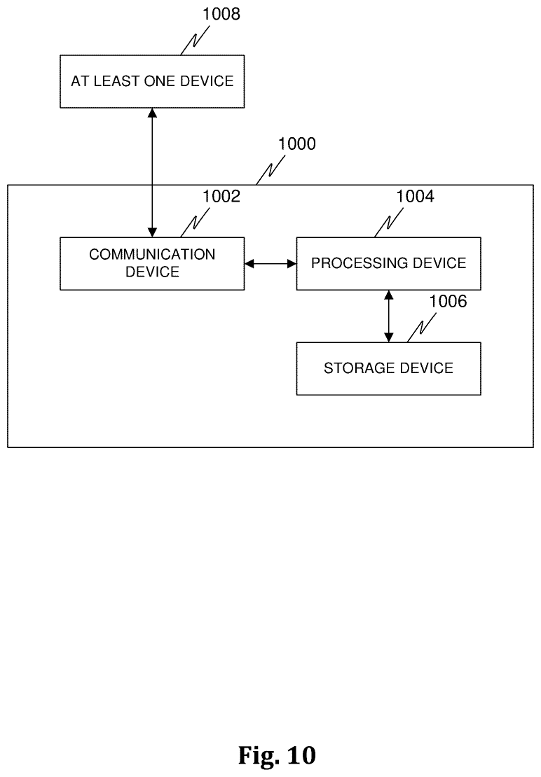

FIG. 10 illustrates a block diagram of a system 1000 for facilitating situation awareness for a physical activity, in accordance with some embodiments. Accordingly, the system 1000 may include a communication device 1002 which may be configured for receiving one or more content data from one or more content source devices. Further, the one or more content data represent one or more contents associated with one or more physical activities. Further, the system 1000 may include a processing device 1004 communicatively coupled with the communication device 1002. Further, the processing device 1004 may be configured for identifying a situation data using one or more temporal artificial intelligence (AI) models based on the one or more content data. Further, the situation may data represent one or more situations associated with the one or more physical activities. Further, the processing device 1004 may be configured for generating an identifier data based on the situation data. Further, the identifier data represents one or more identifiers for the one or more situations. Further, the one or more identifiers represent a position of the one or more situations in a sequence corresponding to the one or more physical activities. Further, the system 1000 may include a storage device 1006 communicatively coupled with the processing device 1004. Further, the storage device 1006 may be configured for storing each of the one or more content data and the identifier data.

Further, in some embodiments, the identifying of the situation data may include identifying an event data representing one or more events associated with the one or more physical activities using the one or more temporal AI models. Further, the identifying of situation data may include identifying a phase data representing one or more phases associated with the one or more events. Further, the one or more identifiers include one or more phase-based identifiers. Further, the generating of the identifier data includes generating a phase-based identifier data based on the identifying of the phase data. Further, the phase-based identifier data represents the one or more phase-based identifiers associated with the one or more physical activities.

Further, in some embodiments, the one or more physical activities may be associated with one or more states of one or more entities. Further, the processing device 1004 may be further configured for evaluating the situation data. Further, the processing device 1004 may be further configured for determining one or more three-dimensional spatial information for the one or more entities relative to the one or more situations based on the situation data. Further, the processing device 1004 may be further configured for generating one or more feedback data based on the evaluating of the situation data and the determining of the one or more three-dimensional spatial information. Further, the one or more feedback data represents one or more feedback associated with the one or more entities relative to the one or more physical activities. Further, the communication device 1002 may be further configured for transmitting the one or more feedback data to one or more devices 1008.

Further, in some embodiments, the one or more content data may include a video stream data representing one or more video streams associated with the one or more physical activities. Further, the one or more feedbacks may include one or more positional correction information associated with the one or more entities relative to the one or more physical activities. Further, the processing device 1004 may be further configured for generating an overlay content data based on one or more positional correction information. Further, the overlay content data represents one or more overlay contents. Further, the one or more content data may include a video stream data representing one or more video streams associated with the one or more physical activities. Further, the processing device 1004 may be further configured for overlaying the one or more overlay contents onto the one or more video streams. Further, the one or more content data may include a video stream data representing one or more video streams associated with the one or more physical activities. Further, the processing device 1004 may be further configured for generating an overlaid video stream data based on the overlaying. Further, the transmitting of the one or more feedback data includes transmitting the overlaid video stream data to the one or more devices 1008.

Further, in some embodiments, the storage device 1006 may be further configured for retrieving one or more reference data for the one or more situations based on the evaluating of the situation data. Further, the processing device 1004 may be further configured for analyzing each of the situation data and the one or more reference data. Further, the processing device 1004 may be further configured for computing one or more performance metrics for the one or more entities based on the analyzing of each of the situation data and the one or more reference data. Further, the generating of the one or more feedback data may be further based on the one or more performance metrics.

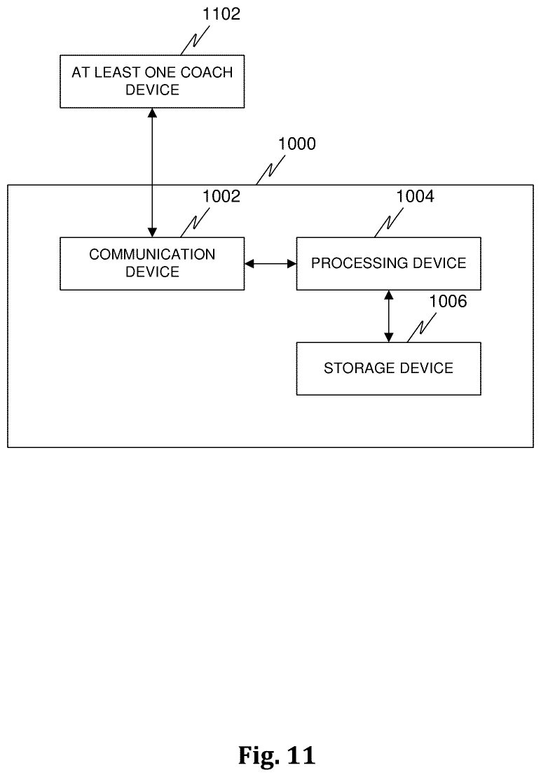

FIG. 11 illustrates a block diagram of the system 1000 for facilitating situation awareness for a physical activity, including at least one coach device 1102, in accordance with some embodiments. Further, in some embodiments, the one or more devices 1008 include one or more coach devices 1102 associated with one or more coaches. Further, the transmitting of the one or more feedback data to the one or more devices 1008 includes transmitting the one or more feedback data to the one or more coach devices 1102. Further, the one or more coach devices 1102 include a coach-side presentation device which may be configured for presenting the one or more feedback to the one or more coaches. Further, the one or more coach devices 1102 further include a coach-side input device which may be configured for generating an input data representing one or more inputs relative to the one or more feedbacks. Further, the one or more coach devices 1102 further include a coach-side communication device, which may be configured for transmitting the input data to the communication device 1002.

In some embodiments, the one or more content data includes one or more sensor data associated with one or more sensor devices, which may be configured for monitoring the one or more physical activities. Further, the processing device 1004 may be further configured for analyzing the one or more sensor data. Further, the determining of the one or more three-dimensional spatial information for the one or more entities may be further based on the analyzing of the one or more sensor data.

In some embodiments, the time stamp data may include one or more of a time-coded chapter data representing the time value associated with one or more chapters associated with the one or more physical activities and a time-coded marker data representing one or more time-coded markers for the one or more situations relative to the one or more physical activities.

Further, in some embodiments, the processing device 1004 may be further configured for identifying one or more equipment data based on the situation data. Further, the one or more equipment data represents one or more equipment associated with the one or more entities relative to the one or more physical activities. Further, the processing device 1004 may be further configured for determining one or more information of the one or more equipment. Further, the generating of the one or more feedback data may be further based on the determining of the one or more information of the one or more equipment.

Further, in some embodiments, the one or more situations may include two or more situations. Further, the processing device 1004 may be further configured for determining a temporal order associated with the two or more situations using the one or more temporal AI models based on the situation data. Further, the processing device 1004 may be further configured for analyzing the temporal order. Further, the generating of the identifier data may be further based on the analyzing of the temporal order.

In some embodiments, the one or more temporal AI models includes one or more of one or more recurrent neural networks (RNN) and one or more transformer-based models. Further, the one or more RNNS include one or more of a long short-term memory (LSTM) and a gated recurrent unit (GRU).

In some embodiments, the one or more physical activities include one or more sporting events. Further, the one or more entities include one or more athletes associated with the one or more sporting events.

In some embodiments, the one or more sporting events include one or more of football, hockey, basketball, and golf.

In some embodiments, the one or more events relative to the basketball include one or more of a jump shot, a free throw, and a layup. Further, the one or more events relative to the football include one or more of a pass, a tackle, and a block performed by the one or more athletes.

In some embodiments, the one or more phases relative to the basketball includes one or more of a transfer phase representing moving of a ball into one or more positions of a shot by the one or more athletes, a pocket phase representing aligning of the ball with a shoulder level of the one or more athletes for optimal release of the ball, and a release phase representing an execution of the shot with one or more controlled wrist movements.

In some embodiments, the one or more phases relative to the football include one or more of a pre-pass setup phase, a release phase, and a follow-through phase.

In some embodiments, the one or more phases related to the hockey include one or more of a stick preparation phase, a puck release phase, and a recovery phase.

In some embodiments, the method 300 may further include analyzing, using the processing device 1004, the one or more content data using the one or more temporal AI models. Further, the identifying of the situation data may be further based on the analyzing of the one or more content data.

In some embodiments, the method 400 may further include storing, using the storage device 1006, the one or more feedback data in association with each of the one or more content data and the situation data.

In some embodiments, the one or more positional correction information includes one or more of a joint angle correction information representing a correction of one or more joint angles, a movement pattern correction information representing a correction of one or more movement patterns, and a body alignment correction information representing a correction of one or more body alignments.

In some embodiments, the one or more sensor devices include one or more motion sensors. Further, the one or more motion sensors includes one or more of an athlete motion sensor which may be configured for tracking one or more athlete movements associated with the one or more athletes during the one or more physical activities and an equipment motion sensor which may be configured for tracking one or more equipment behaviors associated with the one or more equipment associated with the one or more athletes.

In some embodiments, the one or more performance metrics include a fatigue metric representing a fatigue scenario of the one or more entities during the one or more physical activities. Further, the generating of the one or more feedback data may be further based on the fatigue metric.

In some embodiments, the one or more states include one or more of a moving state, a still state, and a posture of the one or more entities.

In some embodiments, the one or more three-dimensional spatial information includes one or more of a guarded position and an unguarded position of the one or more entities relative to the one or more physical activities.

In some embodiments, the generating of the one or more feedback data includes generating the one or more feedback data using the one or more temporal AI models.

In some embodiments, the determining of the one or more information of the one or more equipment includes one or more of determining one or more orientations of the one or more equipment relative to the one or more entities and determining one or more orientations of the one or more equipment relative to an environment associated with the one or more physical activities.

In some embodiments, the one or more equipment includes one or more of a golf club, a hockey stick, and a basketball.

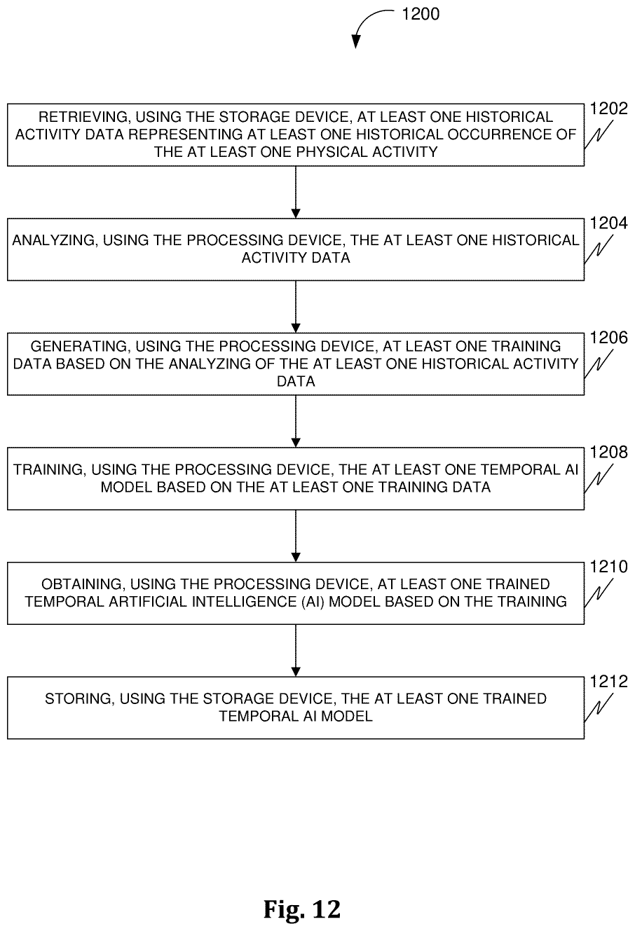

FIG. 12 illustrates a flowchart of a method 1200 for facilitating situation awareness for a physical activity including obtaining, using the processing device 1004, at least one trained temporal artificial intelligence (AI) model, in accordance with some embodiments. Further, in some embodiments, the method 1200 further may include a step 1202 of retrieving, using the storage device 1006, one or more historical activity data representing one or more historical occurrences of the one or more physical activities. Further, in some embodiments, the method 1200 further may include a step 1204 of analyzing, using the processing device 1004, the one or more historical activity data. Further, in some embodiments, the method 1200 further may include a step 1206 of generating, using the processing device 1004, one or more training data based on the analyzing of the one or more historical activity data. Further, in some embodiments, the method 1200 further may include a step 1208 of training, using the processing device 1004, the one or more temporal AI models based on the one or more training data. Further, in some embodiments, the method 1200 further may include a step 1210 of obtaining, using the processing device 1004, one or more trained temporal artificial intelligence (AI) models based on the training. Further, in some embodiments, the method 1200 further may include a step 1212 of storing, using the storage device 1006, the one or more trained temporal AI models.

In some embodiments, the method 1200 may further include obtaining, using the processing device 1004, a temporal data corresponding to the one or more physical activities. Further, the generating of the one or more training data may be further based on the temporal data.

In some embodiments, the generating of the one or more feedback data includes generating the one or more feedback data using one or more explainability artificial intelligence (AI) models.

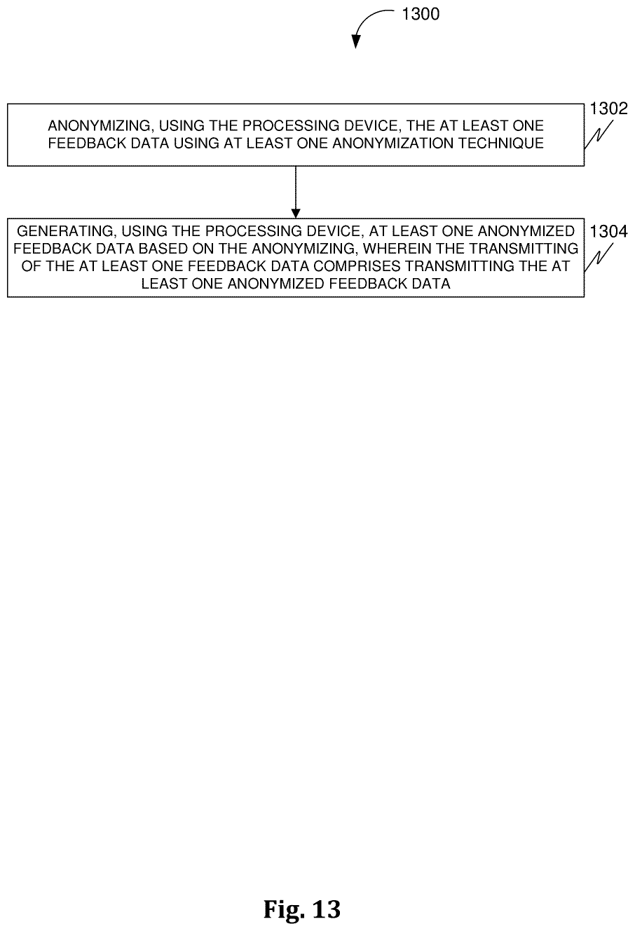

FIG. 13 illustrates a flowchart of a method 1300 for facilitating situation awareness for a physical activity, including generating, using the processing device 1004, at least one anonymized feedback data, in accordance with some embodiments. Further, in some embodiments, the method 1300 further may include a step 1302 of anonymizing, using the processing device 1004, the one or more feedback data using one or more anonymization techniques. Further, in some embodiments, the method 1300 further may include a step 1304 of generating, using the processing device 1004, one or more anonymized feedback data based on the anonymizing. Further, the transmitting of the one or more feedback data includes transmitting the one or more anonymized feedback data.

In some embodiments, the method 1200 may further include obtaining, using the processing device 1004, one or more synthetic data representing one or more rare scenarios associated with the one or more physical activities. Further, the generating of the one or more training data may be further based on the one or more synthetic data. Further, the one or more synthetic data includes one or more of a unique biomechanical pattern and an environmental condition associated with the one or more physical activities.

In some embodiments, each of the receiving of the one or more content data and the transmitting of the one or more feedback data may be based on a real-time communication protocol.

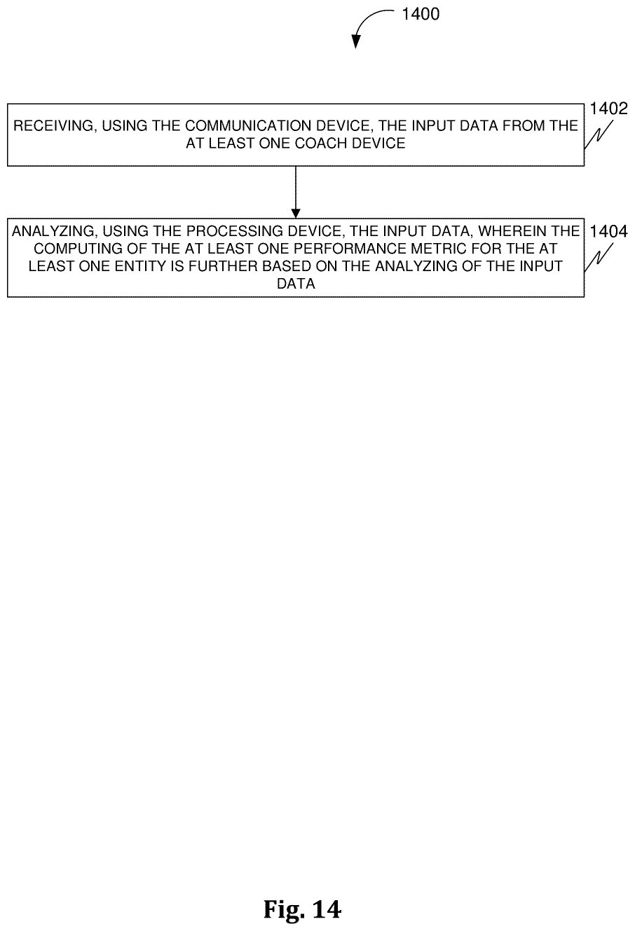

FIG. 14 illustrates a flowchart of a method 1400 for facilitating situation awareness for a physical activity, including analyzing, using the processing device 1004, the input data, in accordance with some embodiments. Further, in some embodiments, the method 1400 further may include a step 1402 of receiving, using the communication device 1002, the input data from the one or more coach devices 1102. Further, in some embodiments, the method 1400 further may include a step 1404 of analyzing, using the processing device 1004, the input data. Further, the computing of the one or more performance metrics for the one or more entities may be further based on the analyzing of the input data.

In some embodiments, the one or more phases include two or more phases. Further, each of the two or more phases corresponds to a time period.

In some embodiments, the temporal data includes a three-dimensional pose data. Further, the three-dimensional pose data includes one or more of a three-dimensional spatial position data and a three-dimensional posture data.

In some embodiments, the one or more situations represent one or more of a part of the one or more physical activities, an environmental condition associated with the one or more physical activities, and an athlete condition associated with the one or more athletes performing the one or more physical activities.

In some embodiments, the one or more performance metrics correspond to one or more characteristics of the one or more physical activities.

In some embodiments, the generating of the one or more feedback data includes generating of one or more second feedback data. Further, the one or more feedback data may be associated with a first time period, and the one or more second feedback data associated with a second time period. Further, the second time period occurs later than the first time period. Further, the method 1200 further includes analyzing, using the processing device 1004, the one or more feedback data. Further, the generating of the one or more second feedback data may be further based on the analyzing of the one or more feedback data.

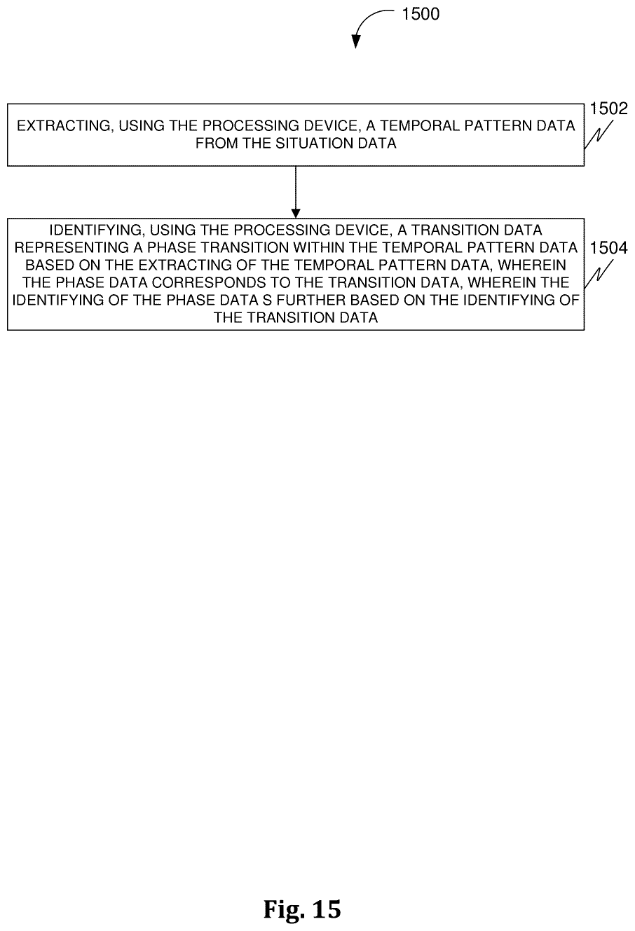

FIG. 15 illustrates a flowchart of a method 1500 for facilitating situation awareness for a physical activity including identifying, using the processing device 1004, a transition data representing a phase transition within the temporal pattern data, in accordance with some embodiments. Further, in some embodiments, the method 1500 further may include a step 1502 of extracting, using the processing device 1004, a temporal pattern data from the situation data. Further, in some embodiments, the method 1500 further may include a step 1504 of identifying, using the processing device 1004, a transition data representing a phase transition within the temporal pattern data based on the extracting of the temporal pattern data. Further, the phase data corresponds to the transition data. Further, the identifying of the phase data is further based on the identifying of the transition data.

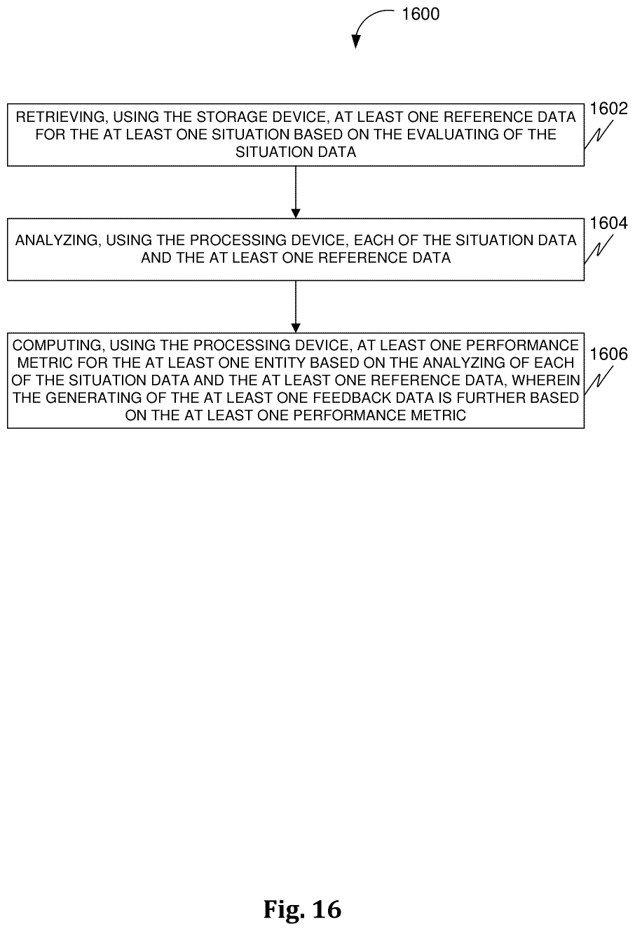

FIG. 16 illustrates a flowchart of a method 1600 for facilitating situation awareness for a physical activity including computing, using the processing device 1004, at least one performance metric for the at least one entity, in accordance with some embodiments. Further, in some embodiments, the method 1600 further may include a step 1602 of retrieving, using the storage device 1006, one or more reference data for the one or more situations based on the evaluating of the situation data. Further, in some embodiments, the method 1600 further may include a step 1604 of analyzing, using the processing device 1004, each of the situation data and the one or more reference data. Further, in some embodiments, the method 1600 further may include a step 1606 of computing, using the processing device 1004, one or more performance metrics for the one or more entities based on the analyzing of each of the situation data and the one or more reference data. Further, the generating of the one or more feedback data may be further based on the one or more performance metrics.

Further, the method 400 may include a step of determining, using the processing device 1004, a situation characteristic data based on the situation data. Further, the situation characteristic data represents one or more situation-based characteristics associated with the one or more situations. Further, the generating of the identifier data is further based on the situation characteristic data. Further, the one or more situation-based characteristic may include one or more of a start time, an end time, and one or more phase boundaries associated with the one or more situations.

In some embodiments, the reference data includes a three-dimensional reference data. Further, the method 1600 further includes correlating, using the processing device 1004, the one or more three-dimensional spatial information with the three-dimensional reference data. Further, the generating of the one or more feedback data may be further based on the correlating of the one or more three-dimensional spatial information with the three-dimensional reference data.

In some embodiments, the method 1600 may further include generating, using the processing device 1004, a comparison result data based on the analyzing of each of the situation data and the one or more reference data. Further, the comparison result data represents a deviation between the one or more reference data and the situation data. Further, the one or more feedback data includes the comparison result data.

In some embodiments, the method 400 may further include analyzing, using the processing device 1004, the one or more three-dimensional spatial information using the one or more temporal AI models. Further, the generating of the one or more feedback data may be further based on the analyzing of the one or more spatial information using the one or more temporal AI models.

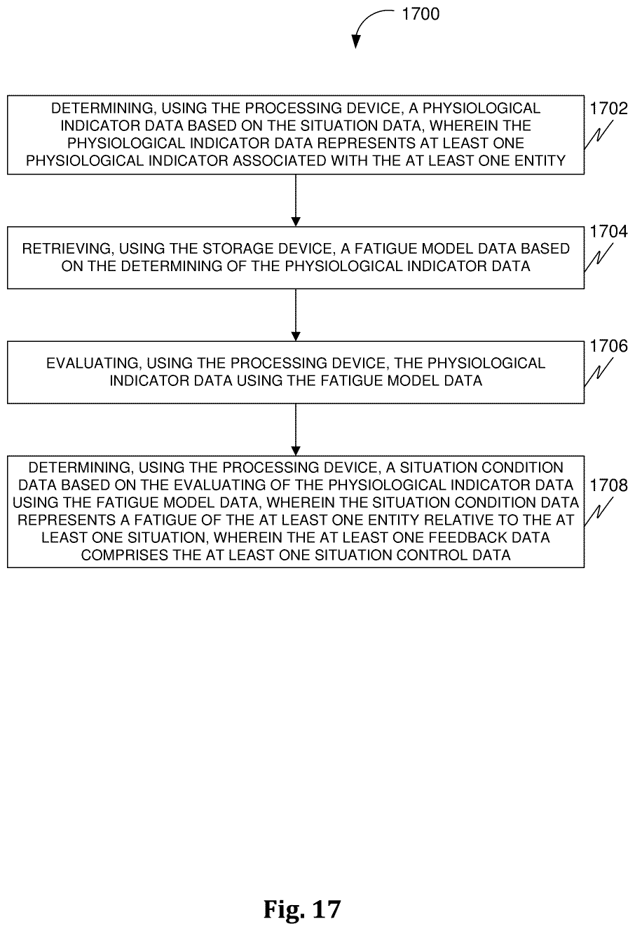

FIG. 17 illustrates a flowchart of a method 1700 for facilitating situation awareness for a physical activity including determining, using the processing device 1004, a situation condition data, in accordance with some embodiments. Further, in some embodiments, the method 1700 further may include a step 1702 of determining, using the processing device 1004, a physiological indicator data based on the situation data. Further, the physiological indicator data represents one or more physiological indicators associated with the one or more entities. Further, in some embodiments, the method 1700 further may include a step 1704 of retrieving, using the storage device 1006, a fatigue model data based on the determining of the physiological indicator data. Further, in some embodiments, the method 1700 further may include a step 1706 of evaluating, using the processing device 1004, the physiological indicator data using the fatigue model data. Further, in some embodiments, the method 1700 further may include a step 1708 of determining, using the processing device 1004, a situation condition data based on the evaluating of the physiological indicator data using the fatigue model data. Further, the situation condition data represents a fatigue of the one or more entities relative to the one or more situations. Further, the one or more feedback data includes the situation condition data.

FIG. 18 illustrates a flowchart of a method 1800 for facilitating situation awareness for a physical activity including generating, using the processing device 1004, at least one enhanced content data, in accordance with some embodiments. Further, in some embodiments, the one or more content data further corresponds to a visual sequence of one or more physical activities. Further, the method 1800 further may include a step 1802 of embedding, using the processing device 1004, the one or more identifiers within the visual sequence. Further, the method 1800 further may include a step 1804 of generating, using the processing device 1004, one or more enhanced content data based on the embedding. Further, the one or more enhanced content data represents the one or more contents embedded with the one or more identifiers. Further, the method 1800 further may include a step 1806 of transmitting, using the communication device 1002, the one or more enhanced content data to the one or more devices 1008. Further, the method 1800 further may include a step 1808 of storing, using the storage device 1006, the one or more enhanced content data.

Further, the one or more situations may include one or more of an activity label associated with the one or more physical activities, a phase within the one or more physical activities, and a state/context for the one or more physical activities. Further, the activity label may include one or more of a jump shot label, a free throw label, and a pass label. Further, the phase may include one or more of a transfer phase, a pocket phase, and a release phase. Further, the state/context may include one or more of a guarded state of the one or more entities, an unguarded state of the one or more entities, a fatigue associated with the one or more entities, a posture state of the one or more entities, a pressure on the of the one or more entities, and an environmental condition during the one or more physical activities.

Further, the computing of the one or more performance metrics may include computing of the one or more performance metrics using the one or more temporal AI models. Further, the computing of the one or more performance metrics using the one or more temporal AI models may include determining one or more confidence scores for the one or more situations using the one or more temporal AI models based on the analyzing of each of the situation data and the one or more reference data. Further, the one or more confidence scores may include a low confidence score and a high confidence score. Further, the generating of the one or more feedback data is further based on the one or more confidence scores comprising the high confidence score.

Further, the one or more identifiers may include one or more of a metadata object, a watermark, an overlay tag, and an event marker associated with the one or more physical activities.

Further, the method 400 may include a step of transmitting, using the communication device 1002, the identifier data in association with the one or more content data to the one or more devices 1008.

Further, the one or more devices 1008 may include a presentation device configured for presenting the one or more enhanced content data. Further, the one or more devices 1008 may include an input device configured for generating a selection data representing a selection relative to the one or more identifiers embedded on the one or more contents associated with the one or more physical activities. Further, the one or more devices 1008 may include a processor configured for modifying the presenting of the one or more enhanced content data based on the selection data. Further, the processor may be further configured for generating one or more modified enhanced content data based on the modifying. Further, the presentation device may be further configured for presenting the one or more modified enhanced content data.

Further, the presenting of the one or more enhanced content data corresponds to a playback of the one or more contents embedded with the one or more identifiers.

In some embodiments, the one or more temporal AI models may be configured for determining one or more activity labels for the one or more situations from two or more activity labels. Further, the identifying of the activity data may be further based on the one or more activity labels.

In some embodiments, the two or more activity labels correspond to two or more sport activities.

In some embodiments, the identifying of the transition data includes identifying of the transition data using one or more of an attention-matrix signal, a hidden-state delta, and a velocity derivative of a three-dimensional pose landmark associated with the one or more physical activities.

In some embodiments, the generating of the one or more training data based on the analyzing of the one or more historical activity data facilitates personalization of the one or more temporal AI models for the at least one or more physical activities and the one or more entities associated with the one or more physical activities.

In some embodiments, the method 500 may further include a step of generating, using the processing device 1004, a preliminary situation summary data based on the situation data. Further, the preliminary situation summary data represents one or more preliminary situation summaries for the one or more situations. Further, the transmitting of the one or more feedback data includes transmitting the preliminary situation summary data to the one or more devices.

In some embodiments, the identifying of the activity data may be further conditioned on one or more of a contextual metadata and a prompt text describing the one or more physical activities.

In some embodiments, the one or more temporal AI models may be configured for consuming one or more multimodal inputs. Further, the one or more multimodal inputs include one or more of an IMU data, a depth data, an audio, and a camera configuration data associated with the one or more physical activities.

FIG. 19 illustrates a user device 1902 configured for presenting the one or more phases 1904 associated with the one or more physical activities, in accordance with some embodiments. Further, the one or more devices 1008 may include the user device 1902. Further, the user device 1902 may be configured for presenting a feedback data 1910. Further, the one or more phases 1904 may represent one or more states of an athlete 1806. Further, the user device 1902 may be configured for presenting a score data 1908 associated with the one or more phases 1904.

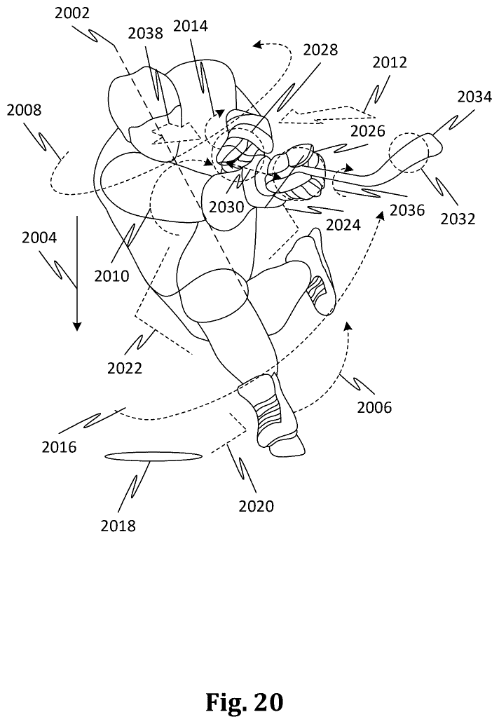

FIG. 20 illustrates a spatial-temporal mechanics of a player playing hockey, in accordance with some embodiments. Further, the spatial-temporal mechanics represents center of balance 2002, transfer of weight 2004, outside leg action 2006, shoulder rotation 2008, rotation of bottom arm 2010, pull of top arm (flexion) 2012, rotation of top hand 2014, stick path 2016, puck position 2018, ankle flexion inside leg 2020, knee flexion inside leg 2022, hip flexion inside leg 2024, grip bottom hand 2026, grip top hand 2028, width of hands 2030, puck impact point 2032, tip control (to target) 2034, shaft rotation (⅛-¼ turn) 2036, targeting—focus 2038.

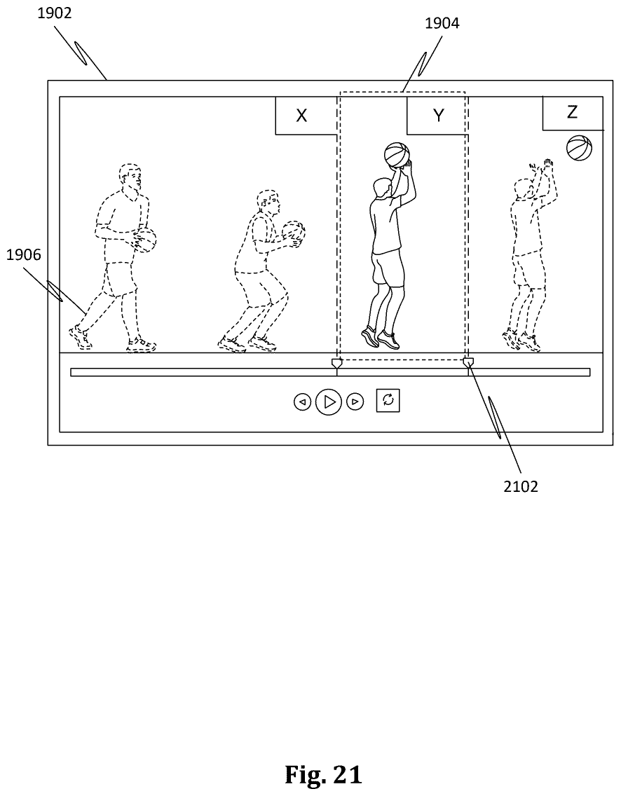

FIG. 21 illustrates the embedding of the one or more identifiers within the visual sequence of the one or more physical activities, in accordance with some embodiments. Further, the one or more identifiers may include one or more markers 2102.

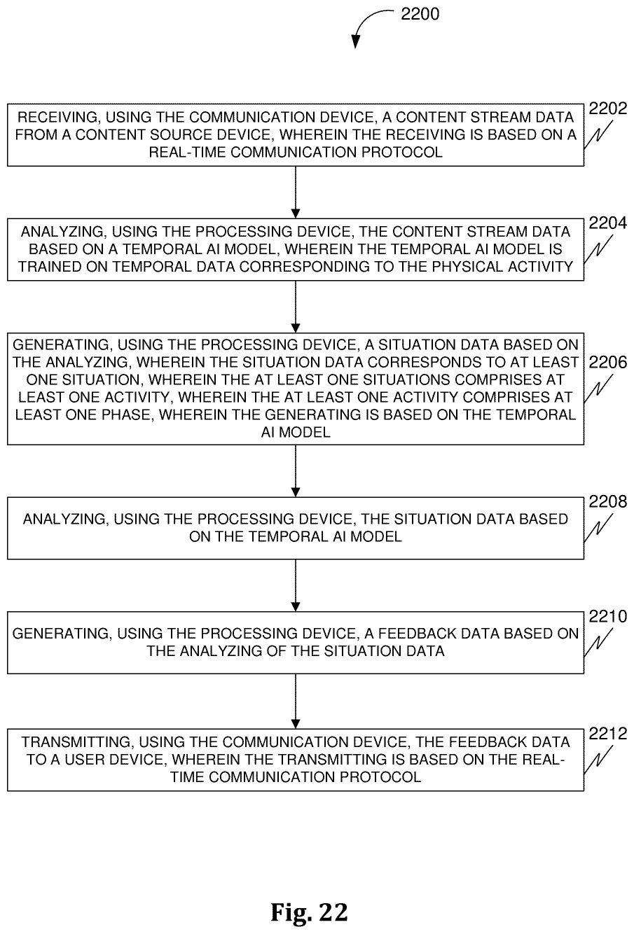

FIG. 22 illustrates a flowchart of a method 2200 of provisioning a contextual feedback of a physical activity, in accordance with some embodiments. Accordingly, the method 2200 may include a step 2202 of receiving, using a communication device 1002, a content stream data from a content source device. Further, the receiving may be based on a real-time communication protocol. Further, the method 2200 may include a step 2204 of analyzing, using a processing device 1004, the content stream data based on a temporal AI model. Further, the temporal AI model may be trained on temporal data corresponding to the physical activity. Further, the method 2200 may include a step 2206 of generating, using the processing device 1004, a situation data based on the analyzing. Further, the situation data corresponds to one or more situations. Further, the one or more situations include one or more activities. Further, the one or more activities include one or more phases. Further, the generating may be based on the temporal AI model. Further, the method 2200 may include a step 2208 of analyzing, using the processing device 1004, the situation data based on the temporal AI model. Further, the method 2200 may include a step 2210 of generating, using the processing device 1004, a feedback data based on the analyzing of the situation data. Further, the method 2200 may include a step 2212 of transmitting, using the communication device 1002, the feedback data to a user device. Further, the transmitting may be based on the real-time communication protocol.

In some embodiments, the situation data includes an activity data corresponding to the one or more activities. Further, the one or more activities may be a part of the physical activity.

In some embodiments, the activity data includes a phase data corresponding to the one or more phases. Further, the one or more phases correspond to a time period.

In some embodiments, the content stream data includes a content data representing a content and a sequential identifier data representing a sequence position of the content data in a sequence corresponding to the physical activity.

In some embodiments, the temporal data includes to a three-dimensional spatial data. Further, the three-dimensional pose data includes one or more of a three dimensional spatial position data and a three dimensional posture data.

In some embodiments, the one or more situations represent one or more of a part of the physical activity, an environmental condition associated with the physical activity and an athlete condition associated with an athlete performing the physical activity.

In some embodiments, the feedback data includes a metric corresponding to a plurality of characteristics of the one or more activities. Further, the generating of the feedback data includes comparing the metric with a reference metric. Further, the reference metric corresponds to the plurality of characteristics.

In some embodiments, the feedback data represents one or more of a progress and a regression in the performance of the one or more activities.

In some embodiments, the metric may further correspond to a situation characteristic. Further, the situation characteristic corresponds to the one or more situations. Further, the reference metric corresponds to the situation characteristic.

In some embodiments, the physical activity corresponds to a sport. Further, the physical activity may be associated with two or more objects. Further, the two or more objects include an athlete performing the physical activity and a sport object. Further, the plurality of characteristics includes an athlete characteristic and a sport object characteristic. Further, athlete characteristic corresponds to the athlete. Further, the sport object characteristic corresponds to the sport object. Further, the sport object characteristic may be based on the athlete characteristic.

Further, the communication device 1002 may be configured for receiving a content stream data from a content source device. Further, the receiving may be based on a real-time communication protocol. Further, the communication device 1002 may be configured for transmitting a feedback data to a user device. Further, the transmitting may be based on the real-time communication protocol. Further, the processing device 1004 may be configured for analyzing the content stream data based on a temporal AI model. Further, the temporal AI model may be trained on temporal data corresponding to the physical activity. Further, the processing device 1004 may be configured for generating a situation data based on the analyzing. Further, the situation data corresponds to one or more situations. Further, the one or more situations include one or more activities. Further, the one or more activities include one or more phases. Further, the generating may be based on the temporal AI model. Further, the processing device 1004 may be configured for analyzing the situation data based on the temporal AI model. Further, the processing device 1004 may be configured for generating the feedback data based on the analyzing of the situation data.

In some embodiments, the situation data includes an activity data corresponding to the one or more activities. Further, the one or more activities may be a part of the physical activity.

In some embodiments, the activity data includes a phase data corresponding to the one or more phases. Further, the one or more phases correspond to a time period.

In some embodiments, the content stream data includes a content data representing a content and a sequential identifier data representing a sequence position of the content data in a sequence corresponding to the physical activity.

In some embodiments, the temporal data includes to a three-dimensional spatial data. Further, the three-dimensional pose data includes one or more of a three-dimensional spatial position data and a three-dimensional posture data.

In some embodiments, the one or more situations represent one or more of a part of the physical activity, an environmental condition associated with the physical activity, and an athlete condition associated with an athlete performing the physical activity.

In some embodiments, the feedback data includes a metric corresponding to a characteristic of the one or more activities. Further, the generating of the feedback data includes comparing the metric with a reference metric. Further, the reference metric corresponds to the characteristic.

In some embodiments, the feedback data represents one or more of a progress and a regression in the performance of the one or more activities.

In some embodiments, the physical activity corresponds to a sport. Further, the physical activity may be associated with two or more objects. Further, the two or more objects include an athlete performing the physical activity and a sports object. Further, the plurality of characteristics includes an athlete characteristic and a sport object characteristic. Further, the athlete characteristic corresponds to the athlete. Further, the sport object characteristic corresponds to the sport object. Further, the sport object characteristic may be based on the athlete characteristic.

In some embodiments, the metric may be further correspond to a situation characteristic. Further, the situation characteristic corresponds to the one or more situations. Further, the reference metric corresponds to the situation characteristic

In some embodiments, the sequential identifier data may be associated with the situation data.

In some embodiments, the situation data includes an activity data corresponding to the one or more activities. Further, the sequential identifier data may be associated with the one or more activity data.

In some embodiments, the activity data includes a phase data corresponding to the one or more phases. Further, the sequential identifier data may be associated with the phase data.

In some embodiments, the situation data includes two or more situation data. Further, the sequential identifier data includes two or more sequential identifier data. Further, the two or more sequential identifier data may be associated with the two or more situation data.

In some embodiments, the two or more situation data includes two or more activity data corresponding to two or more activities. Further, the two or more sequential identifier data may be associated with the two or more activities.

In some embodiments, the two or more activity data includes two or more phase data corresponding to two or more phases. Further, the two or more sequential identifier data may be associated with the two or more phases.

In some embodiments, the sequential identifier data includes a time stamp data representing a time value.

In some embodiments, the sequential identifier data includes a time interval data representing a time interval.

In some embodiments, the two or more sequential identifier data includes two or more time-stamp data representing two or more time values.

In some embodiments, the two or more sequential identifier data includes two or more-time interval data representing two or more time intervals.

In some embodiments, the two or more activities include a first activity and a second activity. Further, the two or more time intervals include a first time interval corresponding to the first activity and a second time interval corresponding to the second activity. Further, the second time interval occurs later than the first time interval.

In some embodiments, the two or more activities include two or more phases that occur over the two or more time intervals. Further, the two or more phases include a first phase and a second phase. Further, the two or more time intervals include a first time interval corresponding to the first phase and a second time interval corresponding to the second phase. Further, the second time interval occurs later than the first time interval.

In some embodiments, the temporal AI model includes a Long Short-Term Memory model.

In some embodiments, the temporal AI model includes a Gated Recurrent Unit model.

In some embodiments, the temporal AI model includes a Recurrent Neural Network model.

In some embodiments, the temporal AI model includes a transformer model.

In some embodiments, the activity data includes two or more activity data. Further, the two or more activity data correspond to two or more activities.

In some embodiments, the physical activity corresponds to Basketball. Further, the two or more activities correspond to one or more of a jump shot, a free throw, and a layup.

In some embodiments, the physical activity corresponds to Football. Further, the two or more activities correspond to one or more of a pass, a tackle, and a blocking event.

In some embodiments, the phase data includes two or more phase data. Further, the two or more phase data corresponds to two or more phases.

In some embodiments, the physical activity corresponds to Basketball. Further, the two or more phases include one or more of a transfer phase, a pocket phase, and a release phase.

In some embodiments, during the transfer phase, an athlete performing the one or more activities moves a ball into a position for performing a shot.

In some embodiments, during the pocket phase, an athlete performing the one or more activities aligns a ball with a shoulder for an optimal release of the ball.

In some embodiments, during the release phase, an athlete performing the one or more activities executes a shot with a wrist movement.

In some embodiments, the physical activity corresponds to Football. Further, the two or more phases include one or more of a pre-pass setup phase, a release phase, and a follow-through phase.

In some embodiments, the physical activity corresponds to Hockey. Further, the two or more phases include one or more of a stick preparation phase, a puck release phase, and a recovery phase.

In some embodiments, the feedback data includes two or more feedback data. Further, the two or more feedback data include two or more metrics. Further, the two or more metrics correspond to two or more characteristics. Further, the two or more characteristics correspond to two or more activities.

In some embodiments, the two or more feedback data may be further based on two or more reference metrics. Further, the two or more reference metrics correspond to the two or more characteristics.

In some embodiments, the two or more activities include a first activity corresponding to a first time period and a second activity corresponding to a second time period. Further, the second time period occurs later than the first time period. Further, the feedback data includes a first feedback data corresponding to the first time period and a second feedback data corresponding to the second time period. Further, the generating of the second feedback data may be based on the first feedback data.

In some embodiments, the first feedback data includes a reference metric. Further, the second feedback data represents one or more of a progress and a regression in the performance of the second activity.

In some embodiments, the activity data includes two or more activity data corresponding to two or more activities. Further, the two or more activities include a first activity occurring at a first time interval and a second activity occurring at a second time interval. Further, the second time interval occurs later than the first time interval. Further, the two or more activity data includes a first activity data corresponding to the first time interval and a second activity corresponding to the second time interval.

In some embodiments, the method 2100 may be the phase data includes two or more phase data corresponding to two or more phases. Further, the two or more phases include a first phase corresponding to a first time period and a second phase corresponding to a second time period. Further, the second time period occurs later than the first time period. Further, the two or more phase data includes a first phase data corresponding to the first time period and a second phase data corresponding to the second time period.

In some embodiments, the athlete characteristic includes a biomechanical characteristic. Further, the biomechanical characteristic corresponds to a mechanics of an athlete.

In some embodiments, the one or more activities correspond to a sport activity. Further, the biomechanical characteristic corresponds to one or more of a joint angle, a movement pattern, and a body alignment of an athlete performing the sports activity.

In some embodiments, the joint angle corresponds to an angle formed between two body parts of the athlete. Further, the two body parts may be linked by a joint.

In some embodiments, the body alignment corresponds to a positioning of body parts of the athlete.

In some embodiments, the movement pattern corresponds to an anatomical movement of one or more body parts of the athlete.

In some embodiments, the physical activity corresponds to a sport activity. Further, the one or more situations correspond to one or more of a guarded shot and an unguarded shot.

In some embodiments, the physical activity corresponds to a Football pass. Further, the one or more situations correspond to one or more of a pressured pass and a relaxed pass.

In some embodiments, the user device may be configured to present the feedback data over a video data.

In some embodiments, the user devices may be configured to execute an application.

In some embodiments, the application may be configured to present the feedback data on a presentation device associated with the user device.

In some embodiments, the feedback data may be used to provide a training to an athlete performing the activity.

In some embodiments, the feedback data includes a suggestion to improve a performance of an athlete performing the one or more activities.

In some embodiments, the physical activity corresponds to Basketball. Further, the one or more phases correspond to a basketball release phase. Further, the suggestion corresponds to correcting a wrist angle of an athlete performing the one or more activities.

In some embodiments, the physical activity corresponds to Football. Further, the one or more phases correspond to a football pass phase. Further, the suggestion corresponds to adjusting a body posture of an athlete performing the one or more phases.

In some embodiments, the generating of the feedback data may be based on a sport type.

In some embodiments, the one or more phases associated with a characteristic.

In some embodiments, the characteristic may be based on one or more of the one or more situations and the one or more activities.

In some embodiments, the real-time communication protocol corresponds to one or more of a WebRTC (or similar), a WebSocket (or similar), a real-time streaming protocol, a real-time transfer protocol and a live-streaming protocol.

In some embodiments, the content stream data includes a video data.

In some embodiments, the content source device corresponds to a camera.

In some embodiments, the user device may be associated with a user.

In some embodiments, the user corresponds to a sport coach.

In some embodiments, the one or more activities may be performed by one or more athletes.

In some embodiments, the temporal AI model may be configured to identify a sequential dependency between two or more activities based on the two or more sequential identifier data.

In some embodiments, the one or more situations correspond to a sports context.

In some embodiments, the one or more activities may be performed by an athlete. Further, the one or more situations correspond to an athlete fatigue.

In some embodiments, the athlete fatigue corresponds to a fatigue experienced by an athlete.

In some embodiments, the fatigue corresponds to one or more of a physical state and a mental state. Further, each of the physical state and the mental state corresponds to a state that affects a performance of the athlete.

In some embodiments, the state corresponds to one or more of a physical tiredness and a mental tiredness.

In some embodiments, the physical activity corresponds to a sport. Further, the analyzing comprises identifying one or more sport-specific activities.

In some embodiments, the sport-specific activity corresponds to one or more of a jump shot, a pass, and a block.

In some embodiments, the generating of the sequence data comprises segmenting the content stream data into one or more phase data.

In some embodiments, the temporal data includes one or more of an audio data, a video data, a time stamp data, an annotated data, and three-dimensional spatial data.

In some embodiments, the environment condition corresponds to a weather condition.

In some embodiments, the two or more metrics may be further correspond to two or more situation characteristics. Further, the two or more situation characteristics correspond to two or more situations.

In some embodiments, the three-dimensional spatial position data represents a three-dimensional position of an athlete performing the physical activity.

In some embodiments, the three-dimensional posture data represents a three-dimensional orientation of an athlete performing the physical activity.

In some embodiments, the sport object characteristic includes a mechanics of a sport object.

In some embodiments, the sport object corresponds to a ball

In some embodiments, the sport object corresponds to a hockey stick,

In some embodiments, the sport object corresponds to a golf club. Further, the golf club may be used to hit a ball in a Golf sport.

In some embodiments, the sport object characteristics correspond to an angle of the golf club in relation to a ground performing the physical activity. In some embodiments, the sport object corresponds to a baseball bat.

Although the invention has been explained in relation to its preferred embodiment, it is to be understood that many other possible modifications and variations can be made without departing from the spirit and scope of the invention as hereinafter claimed.

What is claimed is:

1. A method for facilitating situation awareness for a physical activity, the method comprising:
receiving, using a communication device, at least one content data from at least one content source device, wherein the at least one content data represents at least one content associated with at least one physical activity;
identifying, using a processing device, a situation data using at least one temporal artificial intelligence (AI) model based on the at least one content data, wherein the situation data represents at least one situation associated with the at least one physical activity;
generating, using the processing device, an identifier data based on the situation data, wherein the identifier data represents at least one identifier for the at least one situation, wherein the at least one identifier represents a position of the at least one situation in a sequence corresponding to the at least one physical activity; and
storing, using a storage device, each of the at least one content data and the identifier data.

2. The method of claim 1, wherein the identifying of the situation data comprises:
identifying an event data representing at least one event associated with the at least one physical activity using the at least one temporal AI model; and
identifying a phase data representing at least one phase associated with the at least one event, wherein the at least one identifier comprises at least one phase-based identifier, wherein the generating of the identifier data comprises generating a phase-based identifier data based on the identifying of the phase data, wherein the phase-based identifier data represents the at least one phase-based identifier associated with the at least one physical activity.

3. The method of claim 1, wherein the at least one physical activity is associated with at least one state of at least one entity, wherein the method further comprising:
evaluating, using the processing device, the situation data;
determining, using the processing device, at least one three-dimensional spatial information for the at least one entity relative to the at least one situation based on the situation data;
generating, using the processing device, at least one feedback data based on the evaluating of the situation data and the determining of the at least one three-dimensional spatial information, wherein the at least one feedback data represents at least one feedback associated with the at least one entity relative to the at least one physical activity; and
transmitting, using the communication device, the at least one feedback data to at least one device.

4. The method of claim 3, wherein the at least one content data comprises a video stream data representing at least one video stream associated with the at least one physical activity, wherein the at least one feedback comprises at least one positional correction information associated with the at least one entity relative to the at least one physical activity, wherein the method further comprising:
generating, using the processing device, an overlay content data based on at least one positional correction information, wherein the overlay content data represents at least one overlay content;
overlaying, using the processing device, the at least one overlay content onto the at least one video stream; and
generating, using the processing device, an overlaid video stream data based on the overlaying, wherein the transmitting of the at least one feedback data comprises transmitting the overlaid video stream data to the at least one device.

5. The method of claim 1 further comprising:
extracting, using the processing device, a feature data from the situation data;
retrieving, using the storage device, a stored activity template data based on the extracting of the feature data;
analyzing, using the processing device, the feature data and the stored activity template data; and
identifying, using the processing device, an activity data based on the analyzing of the feature data and the stored activity template data, wherein the identifying of the situation data further comprises identifying the activity data.

6. The method of claim 2, wherein the at least one temporal AI model outputs at least one confidence score associated with at least one of the situation data, the event data, and the phase data, wherein the generating of at least one of the identifier data and the at least one feedback data is gated based on the at least one confidence score.

7. The method of claim 1 further comprising detecting, using the processing device, a start time and an end time for an activity instance within a continuous content stream, wherein the identifier data comprises a timestamp data corresponding to the start time and the end time.

8. The method of claim 1, wherein the at least one temporal AI model comprises at least one of a recurrent neural network, a transformer model, a temporal convolutional network, a graph neural network, and a vision-language model, wherein the at least one temporal AI model consumes a multi-modal input comprising a video and at least one of an IMU data, a depth data, an audio, and a camera configuration metadata.

9. The method of claim 1, wherein the identifier data comprises at least one of a time stamp data representing a time value associated with the at least one situation and a time interval data representing a time interval associated with the at least one situation relative to the at least one content.

10. The method of claim 1, wherein the at least one situation comprises a plurality of situations, wherein the method further comprises:
determining, using the processing device, a temporal order associated with the plurality of situations using the at least one temporal AI model based on the situation data; and
analyzing, using the processing device, the temporal order, wherein the generating of the identifier data is further based on the analyzing of the temporal order.

11. A system for facilitating situation awareness for a physical activity, the system comprising:
a communication device configured for receiving at least one content data from at least one content source device, wherein the at least one content data represents at least one content associated with at least one physical activity;
a processing device communicatively coupled with the communication device, wherein the processing device is configured for:
identifying a situation data using at least one temporal artificial intelligence (AI) model based on the at least one content data, wherein the situation data represents at least one situation associated with the at least one physical activity; and
generating an identifier data based on the situation data, wherein the identifier data represents at least one identifier for the at least one situation, wherein the at least one identifier represents a position of the at least one situation in a sequence corresponding to the at least one physical activity; and
a storage device communicatively coupled with the processing device, wherein the storage device is configured for storing each of the at least one content data and the identifier data.

12. The system of claim 11, wherein the identifying of the situation data comprises:
identifying an event data representing at least one event associated with the at least one physical activity using the at least one temporal AI model; and
identifying a phase data representing at least one phase associated with the at least one event, wherein the at least one identifier comprises at least one phase-based identifier, wherein the generating of the identifier data comprises generating a phase-based identifier data based on the identifying of the phase data, wherein the phase-based identifier data represents the at least one phase-based identifier associated with the at least one physical activity.

13. The system of claim 11, wherein the at least one physical activity is associated with at least one state of at least one entity, wherein the processing device is further configured for:
evaluating the situation data;
determining at least one three-dimensional spatial information for the at least one entity relative to the at least one situation based on the situation data; and
generating at least one feedback data based on the evaluating of the situation data and the determining of the at least one three-dimensional spatial information, wherein the at least one feedback data represents at least one feedback associated with the at least one entity relative to the at least one physical activity, wherein the communication device is further configured for transmitting the at least one feedback data to at least one device.

14. The system of claim 13, wherein the at least one content data comprises a video stream data representing at least one video stream associated with the at least one physical activity, wherein the at least one feedback comprises at least one positional correction information associated with the at least one entity relative to the at least one physical activity, wherein the processing device is further configured for:
generating an overlay content data based on at least one positional correction information, wherein the overlay content data represents at least one overlay content;
overlaying the at least one overlay content onto the at least one video stream; and
generating an overlaid video stream data based on the overlaying, wherein the transmitting of the at least one feedback data comprises transmitting the overlaid video stream data to the at least one device.

15. The system of claim 11, wherein the processing device is further configured for:
extracting a feature data from the situation data;
analyzing the feature data and a stored activity template data; and
identifying an activity data based on the analyzing of the feature data and the stored activity template data, wherein the identifying of the situation data further comprises identifying the activity data, wherein the storage device is further configured for retrieving the stored activity template data based on the extracting of the feature data.

16. The system of claim 12, wherein the at least one temporal AI model outputs at least one confidence score associated with at least one of the situation data, the event data, and the phase data, wherein the generating of at least one of the identifier data and the at least one feedback data is gated based on the at least one confidence score.

17. The system of claim 11, wherein the processing device is further configured for detecting a start time and an end time for an activity instance within a continuous content stream, wherein the identifier data comprises timestamp data corresponding to the start time and the end time.

18. The system of claim 11, wherein the at least one temporal AI model comprises at least one of a recurrent neural network, a transformer model, a temporal convolutional network, a graph neural network, and a vision-language model, wherein the at least one temporal AI model consumes a multi-modal input comprising a video and at least one of an IMU data, a depth data, an audio, and a camera configuration metadata.

19. The system of claim 11, wherein the identifier data comprises at least one of a time stamp data representing a time value associated with the at least one situation and a time interval data representing a time interval associated with the at least one situation relative to the at least one content.

20. The system of claim 11, wherein the at least one situation comprises a plurality of situations, wherein the processing device is further configured for:
determining a temporal order associated with the plurality of situations using the at least one temporal AI model based on the situation data; and
analyzing the temporal order, wherein the generating of the identifier data is further based on the analyzing of the temporal order.
